(19) United States
(12) Patent Application Publication
(10) Pub. No.: US 2014/0212381 A1
(43) Pub. Date: Jul. 31, 2014

(54) MACROCYCLES

(71) Applicant: Scynexis, Inc., Durham, NC (US)

(72) Inventors: Keqiang Li, Cary, NC (US); Michael Robert Peel, Chapel Hill, NC (US)

(73) Assignee: Scynexis, Inc., Durham, NC (US)

(21) Appl. No.: 14/059,300

(22) Filed: Oct. 21, 2013

Related U.S. Application Data
(60) Provisional application No. 61/716,220, filed on Oct. 19, 2012.
(60) Provisional application No. 61/775,191, filed on Mar. 8, 2013.

(51) Int. Cl. C07K 7/64; A61K 38/13; C07C 271/24; C07C 321/28; A61K 38/21; C07C 229/48

(52) U.S. Cl. CPC C07K 7/645; A61K 38/21; C07C 229/48; C07C 271/24; C07C 321/28; A61K 38/13
USPC 424/85.4; 514/3.8; 514/4.3; 514/20.5; 530/321; 560/115; 564/440

(57) ABSTRACT

Cyclosporin derivatives, methods of manufacturing the cyclosporin derivatives and methods for treating subjects infected with certain viruses, including hepatitis virus or HIV by administering the cyclosporin derivatives are described.

### CROSS-REFERENCE TO PRIOR APPLICATIONS

This application claims priority of U.S. Provisional Application Nos. 61/716,220, filed Oct. 19, 2012 and 61/775,191, filed Mar. 8, 2013, each hereby expressly incorporated by reference in its entirety and each assigned to the assignee hereof.

### FIELD

Disclosed herein are cyclosporin derivatives, compositions comprising them, processes for their preparation, intermediates useful in their synthesis, their use as therapeutics as antiviral agents, methods of inhibiting cyclophilins with a selected cyclosporin derivative, and methods of treating a subject having chronic hepatitis C and other viruses involving the use of a selected cyclosporin derivative in combination with interferon and optionally ribavirin.

### BACKGROUND

Cyclosporine A is well known for its immunosuppressive activity and a range of therapeutic uses, including antifungal, anti-parasitic, and anti-inflammatory as well as anti-HIV activity. Cyclosporine A and certain derivatives have been reported as having anti-HCV activity, see Watashi et al., Hepatology, 2003, 38: 1282-1288, Nakagawa et al., Biochem. Biophys. Res. Commun. 2004, 313: 42-7, and Shimotohno and K. Watashi, 2004, American Transplant Congress, Abstract No. 648 (American Journal of Transplantation 2004, Volume 4, Issue s8, Pages 1-653).

### SUMMARY

In one aspect, provided herein are cyclosporine A derivatives in which the 3-Sarcosine position is substituted by a group —S—CH₂C[CH₂(CH₂)ₙ]NR²R³, wherein R² is hydrogen or an alkyl chain having from one to four carbon atoms and, when the alkyl chain has 3 or 4 carbon atoms, the chain is a straight or branched; R³ is an alkyl chain having from one to four carbon atoms and, when the alkyl chain has 3 or 4 carbon atoms the chain is a straight or branched; and n is 1 or 2.

In this aspect, provided herein are compounds of the formula (I):

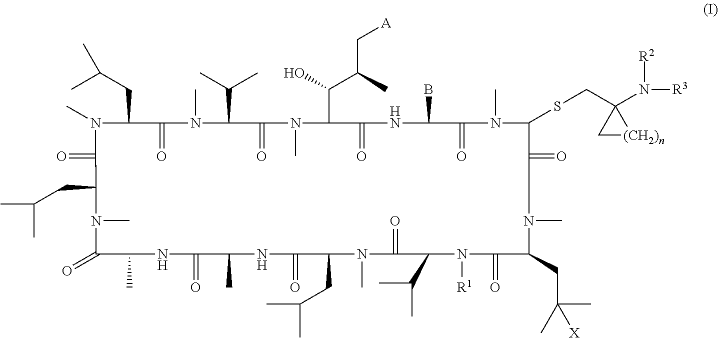

wherein:
A is (E)-CH═CHCH₃ or —CH₂CH₂CH₃;
B is ethyl, 1-hydroxyethyl, isopropyl or n-propyl;
n is 1 or 2;
X is hydrogen or hydroxyl;
   R¹ is hydrogen or straight- or branched-chain alkyl containing from one to four carbon atoms optionally substituted by one or more groups R⁴ which may be the same or different;
R² is hydrogen or an alkyl chain having from one to four carbon atoms and, when the alkyl chain has 3 or 4 carbon atoms, the chain is a straight or branched;
R³ is an alkyl chain having from one to four carbon atoms and, when the alkyl chain has 3 or 4 carbon atoms the chain is a straight or branched; and
R⁴ is phenyl optionally substituted by from one to five groups which may be the same or different selected from the group consisting of alkyl, haloalkyl, halogen, hydroxyl, alkoxy, amino, N alkylamino, N,N dialkylamino, carboxyl and alkoxycarbonyl;
or a pharmaceutically acceptable salt thereof.

In certain cases, the substituents A, B, R² and R³ can contribute to optical and/or stereoisomerism. All such forms are encompassed by exemplary embodiments described herein.

In another aspect, provided are compositions comprising a compound of formula (I) along with a pharmaceutically acceptable excipient, carrier or diluent.

In yet another aspect, provided herein are methods of using a compound of formula (I), or a composition comprising a compound of formula (I), to treat or prevent an infection. Exemplary infections include HCV (Hepatitis C Virus), HBV (Hepatitis B Virus) and HIV (Human Immunodeficiency Virus) infection and others described in detail herein. The methods generally comprise administering to a subject having the virus an amount of the compound or composition effective to treat or prevent the virus.

In still another aspect, provided herein is a compound of formula (I), or a composition comprising a compound of formula (I), for use in therapy.

In another aspect, provided herein is a compound of formula (I), or a composition comprising a compound of formula (I), in the manufacture of a medicament.

In another aspect, provided is a compound of formula (I), or a composition comprising a compound of formula (I), for use in the manufacture of a medicament for treatment or prevention of a virus.

The compounds provided herein, in some aspects of their properties, for example their distribution properties between red blood cells and plasma, and their ability to induce IL29 in HCV-infected cells such as human peripheral blood mononuclear cells, show advantages over known compounds.

### DETAILED DESCRIPTION

### Definitions

When referring to the compounds and complexes disclosed herein, the following terms have the following meanings unless indicated otherwise.

“Cyclosporine” refers to a cyclosporine compound known to those of skill in the art, or a derivative thereof. See, e.g., Ruegger et al., 1976, Helv. Chim. Acta. 59: 1075-92; Borel et al., 1977, Immunology 32: 1017-25; the contents of which are hereby incorporated by reference in their entireties. The compounds of formula (I) are cyclosporine derivatives. Unless noted otherwise, a cyclosporine described herein is a cyclosporine A.

The cyclosporine nomenclature and numbering systems used hereafter are those used by J. Kallen et al., “Cyclosporins: Recent Developments in Biosynthesis, Pharmacology and Biology, and Clinical Applications”, Biotechnology, second edition, H.-J. Rehm and G. Reed, ed., 1997, p 535-591 and are shown below:

| Position | Amino acid in cyclosporine A |
|---|---|
| 1 | N-Methyl-butenyl-threonine (MeBmt) |
| 2 | [alpha]-aminobutyric acid (Abu) |
| 3 | Sarcosine (Sar) |
| 4 | N-Methyl-leucine (MeLeu) |
| 5 | Valine (Val) |
| 6 | N-Methyl-leucine (MeLeu) |
| 7 | Alanine (Ala) |
| 8 | (D)-Alanine ((D)-Ala) |
| 9 | N-Methyl-leucine (Me-Leu) |
| 10 | N-Methyl-leucine (MeLeu) |
| 11 | N-Methylvaline (MeVal) |

where “Bmt” refers to 2(S)-amino-3(R)-hydroxy-4(R)-methyl-6(E)-octenoic acid.

Cyclosporine A is a cyclic nonribosomal peptide of 11 amino acids and contains a single D-amino acid.

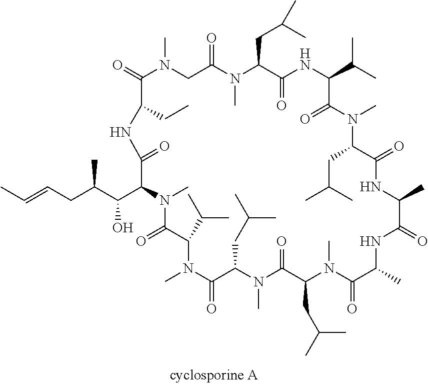 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

This corresponds to the saturated ring of carbon atoms in the compounds of formula (I) as shown below:

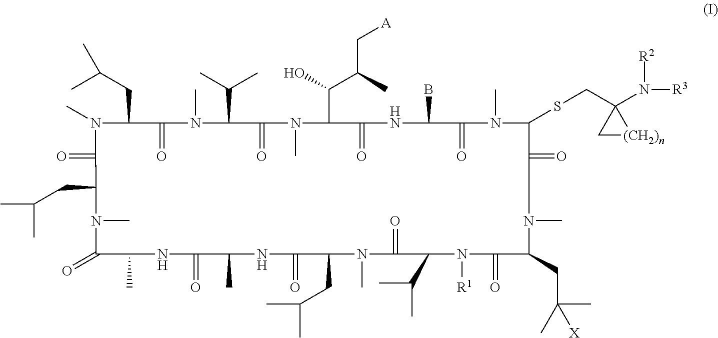

where A, B, R¹, R² and R³ are as defined above.

A “cyclophilin inhibitor” is a compound capable of inhibiting the activity of a cyclophilin. A cyclophilin inhibitor can bind a cyclophilin and inhibit the activity of the cyclophilin. Cyclophilin binding compounds are cyclophilin inhibitors. Exemplary compounds can include cyclosporines that are useful in the treatment of certain indications and exhibit beneficial properties. Such beneficial properties include, for example, interferon-like behavior.

“Alkyl” refers to monovalent saturated aliphatic hydrocarbyl groups having up to 4 carbon atoms. The hydrocarbon chain may be either straight-chained or branched. This term is illustrated by the groups methyl, ethyl, n-propyl, isopropyl, n-butyl, iso-butyl, and tert-butyl.

A “leaving group” refers to a nucleofuge, which is a group that carries away the bonding electron pair when it is displaced by a nucleophile.

“Pharmaceutically acceptable salt” refers to any salt of a compound disclosed herein which retains its biological properties and which is not toxic or otherwise undesirable for pharmaceutical use. Such salts may be derived from a variety of organic and inorganic counter-ions well known in the art. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and like acids.

Salts further include, by way of example only salts of non-toxic organic or inorganic acids, such as hydrohalides, e.g., hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

It is to be understood that compounds having the same molecular formula but differing in the nature or sequence of bonding of their atoms or in the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is designated (R) or (S) according to the rules of Cahn and Prelog (Cahn et al., 1966, Angew. Chem. 78: 413-447, Angew. Chem., Int. Ed. Engl. 5: 385-414 (errata: Angew. Chem., Int. Ed Engl. 5:511); Prelog and Helmchen, 1982, Angew. Chem. 94: 614-631, Angew. Chem. Internal. Ed. Eng. 21: 567-583; Mata and Lobo, 1993, Tetrahedron: Asymmetry 4: 657-668) or can be characterized by the manner in which the molecule rotates the plane of polarized light and is designated dextrorotatory or levorotatory (i.e., as (+)- or (−)-isomers, respectively). A chiral compound can exist as either individual enantiomer or as a mixture thereof. A mixture containing equal proportions of enantiomers is called a “racemic mixture”.

In certain embodiments, the compounds disclosed herein may possess one or more asymmetric centers; such compounds can therefore be produced as the individual (R)- or (S)-enantiomer or as a mixture thereof. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art. In particular embodiments, stereoisomers of the compounds provided herein are depicted upon treatment with base.

In certain embodiments, the compounds disclosed herein are “stereochemically pure”. A stereochemically pure compound has a level of stereochemical purity that would be recognized as “pure” by those of skill in the art. Of course, this level of purity will be less than about 100%. In certain embodiments, “stereochemically pure” designates a compound that is substantially free, i.e. at least about 85% or more, of alternate isomers. In particular embodiments, the compound is at least about 85%, about 90%, about 91%, about 92%, about 93%, about 94%, about 95%, about 96%, about 97%, about 98%, about 99%, about 99.5% or about 99.9% free of other isomers.

“Sarcosine” or “Sar” refers to the amino acid residue known to those of skill in the art having the structure —N(Me)CH₂C(═O)—. Those of skill in the art might recognize sarcosine as N-methyl glycine.

As used herein, the terms “subject” and “patient” are used interchangeably herein. The terms “subject” and “subjects” refer to a primate (e.g., a monkey such as a cynomolgous monkey, a chimpanzee and a human). In one embodiment, the subject is a human.

As used herein, the terms “therapeutic agent” and “therapeutic agents” refer to any agent(s) which can be used in the treatment, management, or amelioration of a disorder or one or more symptoms thereof. In certain embodiments, the term “therapeutic agent” refers to a compound disclosed herein. In certain other embodiments, the term “therapeutic agent” does not refer to a compound disclosed herein. In one embodiment, a therapeutic agent is an agent that is known to be useful for, or has been or is currently being used for the treatment, management, prevention, or amelioration of a disorder or one or more symptoms thereof.

“Therapeutically effective amount” means an amount of a compound or complex or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in one embodiment, to ameliorating a disease or disorder that exists in a subject. In another embodiment, “treating” or “treatment” refers to ameliorating at least one physical parameter or substantially inhibiting a symptom, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” refers to a compound disclosed herein. In one embodiment, a prophylactic agent is an agent which is known to be useful for, or has been or is currently being used to prevent or impede the onset, development, progression and/or severity of a disorder.

As used herein, the terms “prevent”, “preventing” and “prevention” refer to the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, the phrase “prophylactically effective amount” refers to the amount of a therapy (e.g., prophylactic agent) which is sufficient to result in the prevention of the development, recurrence or onset of one or more symptoms associated with a disorder, or to enhance or improve the prophylactic effect(s) of another therapy (e.g., another prophylactic agent).

The term “label” refers to a display of written, printed or graphic matter upon the immediate container of an article, for example, the written material displayed on a vial containing a pharmaceutically active agent.

The term “labeling” refers to all labels and other written, printed or graphic matter upon any article or any of its containers or wrappers or accompanying such article, for example, a package insert or instructional videotapes or DVDs accompanying or associated with a container of a pharmaceutically active agent.

### Compounds

In one embodiment X is hydroxyl.

In one embodiment, A is (E) —CH═CH—CH₃.

In another embodiment, B is ethyl.

In yet another embodiment, n is 1.

In yet another embodiment R¹ is hydrogen or benzyl.

In a further embodiment, R² is methyl or ethyl.

In a still further embodiment, R³ is methyl, ethyl or isopropyl.

In certain embodiments, there are provided compounds of formula (I) in which:
A is (E)-CH═CHCH₃;
B is ethyl;
n is 1 or 2;
R¹ is hydrogen or benzyl; and
R² and R³, which may be the same or different, each are a C₁-C₄ alkyl group.

In one embodiment, the compounds of formula (I) provided herein are selected from the following:

| Compound | Name |
|---|---|
| A | [(R)-[(1-(N,N-dimethylamino)cyclopropyl)methylthio- |
| | Sar]³[4′-hydroxy-N-methylleucine]⁴-cyclosporine A |
| B | [(R)-[(1-(N-methyl-N- |
| | isopropylamino)cyclopropyl)methylthio- |
| | Sar]³[4′-hydroxy-N-methylleucine]⁴-cyclosporine A |
| C | [(R)-[(1-(N,N-dimethylamino)cyclobutyl)methylthio- |
| | Sar]³[4′-hydroxy-N-methylleucine]⁴-cyclosporine A |
| D | [(R)-[(1-(N,N-diethylamino)cyclopropyl)methylthio- |
| | Sar]³[4′-hydroxy-N-methylleucine]⁴-cyclosporine A |
| E | [(R)-[(1-(N-ethyl-N-methylamino)cyclopropyl)methylthio- |
| | Sar]³[4′-hydroxy-N-methylleucine]⁴-cyclosporine A |
| F | [(R)-[(1-(N,N-dimethylamino)cyclobutyl)methylthio- |
| | Sar]³ (N-benzyl)-Val⁵-cyclosporine A. |

The letters A to F are used to identify the above compounds hereafter.

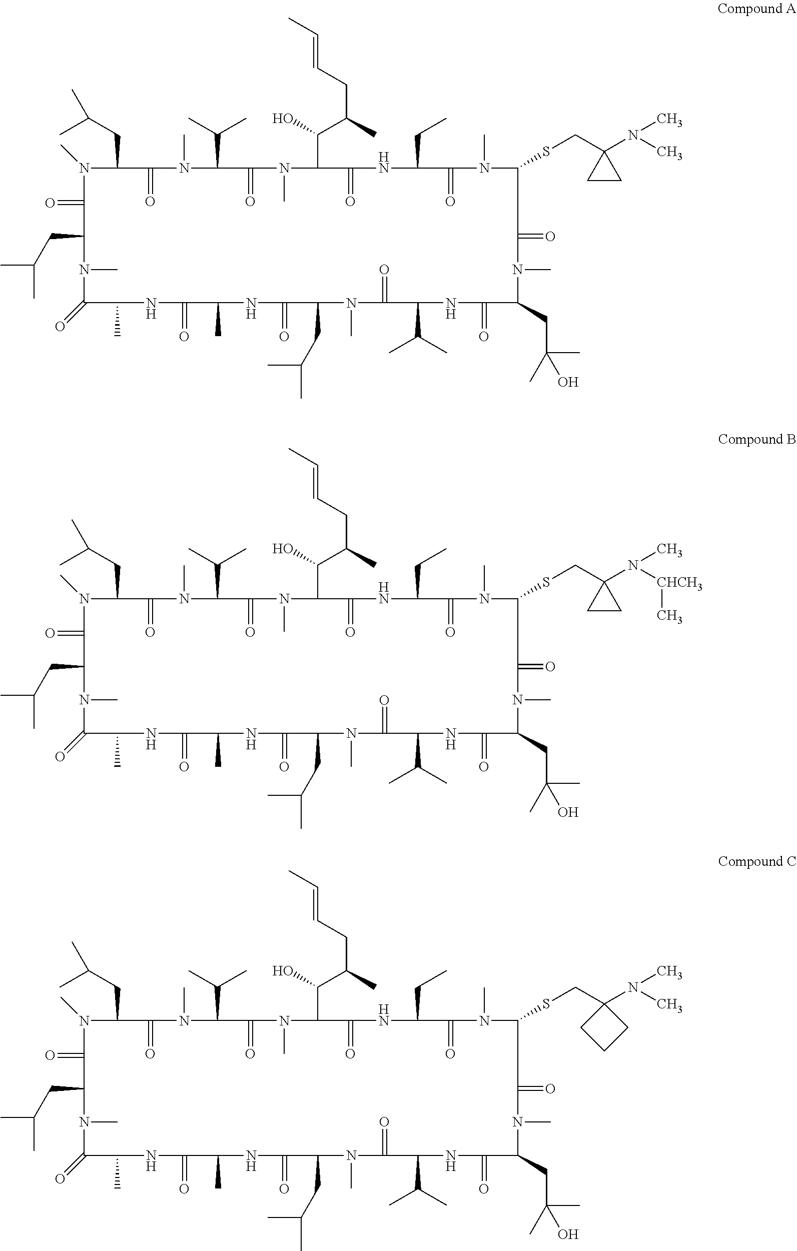

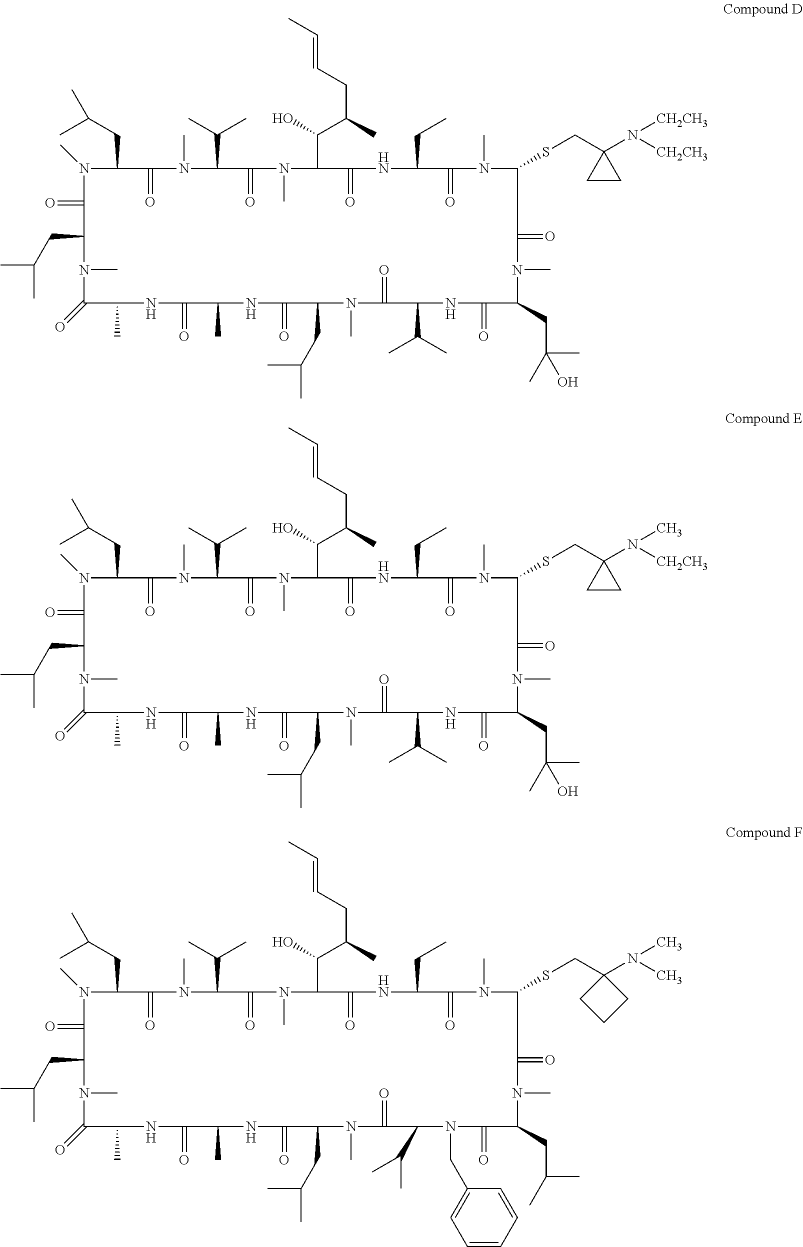

The compounds of formula (I) can be prepared, isolated or obtained by any method apparent to those of skill in the art. Exemplary methods of preparation are described in detail in the examples below.

In one embodiment, compounds of formula (I) may be prepared by the reaction of a compound of formula (II):

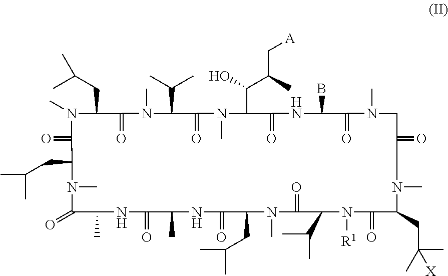

wherein R¹, A and B are as defined above, with a compound of formula (III):

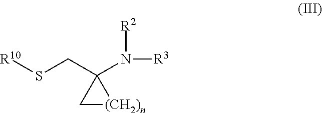

wherein R², R³ and n are as defined above and R¹⁰ is a leaving group, for example a tosylate, mesylate, a quarternary ammonium, or a halide. Compounds of formula (III) are novel and as such form a further embodiment.

Compounds of formula (II) above are known in the literature; see for example European Patent No. 484,281 and WO2009/148615.

Compounds of formula (III) above may be prepared by reacting a compound of formula (IV):

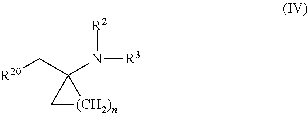

wherein R²⁰ is a halogen (e.g. bromide) with a compound of formula R¹⁰S^{—}X⁺, wherein R¹⁰ is as defined above and X is a cation. Examples of suitable cations include alkaline metals (e.g. sodium and potassium). The reaction is generally performed in an aprotic solvent (such as acetonitrile) and in the presence of a base, such as potassium carbonate. Compounds of formula (IV) are known or made be prepared by the application or adaptation of known methods.

### Pharmaceutical Compositions and Methods of Administration

The compounds of formula (I) used in the methods disclosed herein can be administered in certain embodiments using pharmaceutical compositions including at least one compound of general formula (I), if appropriate in the salt form, either used alone or in the form of a combination with one or more compatible and pharmaceutically acceptable carriers, such as diluents or adjuvants, or with another therapeutic agent. In clinical practice exemplary cyclosporine compounds can be administered by any conventional route, in particular orally, parenterally, rectally or by inhalation (e.g., in the form of aerosols). In one embodiment, the compounds disclosed herein are administered orally.

### Dosage Routes and Forms

Examples of routes of administration include, but are not limited to, oral, parenteral, e.g., intravenous, intradermal, subcutaneous, intramuscular, subcutaneous, buccal, sublingual, inhalation, intranasal, transdermal, topical, transmucosal, intra-tumoral, intra-synovial, and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition can also include a solubilizing agent and a local anesthetic such as lignocaine to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms provided herein will typically vary depending on their use. For example, a dosage form used in the initial treatment of viral infection can contain larger amounts of one or more of the active ingredients it comprises than a dosage form used in the maintenance treatment of the same infection. Similarly, a parenteral dosage form can contain smaller amounts of one or more of the active ingredients it comprises than an oral dosage form used to treat the same disease or disorder. These and other ways in which specific dosage forms encompassed by exemplary embodiments will vary from one another will be readily apparent to those skilled in the art. See, e.g., Remington's Pharmaceutical Sciences, 20^{th} ed., Mack Publishing, Easton Pa. (2000).

### Oral Dosage Forms

Pharmaceutical compositions disclosed herein that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and can be prepared by methods of pharmacy well-known to those skilled in the art. See generally, Remington's Pharmaceutical Sciences, 20^{th} ed., Mack Publishing, Easton Pa. (2000).

Use can be made, as solid compositions for oral administration, of tablets, pills, hard gelatin capsules, powders or granules. In these compositions, the active product is mixed with one or more inert diluents or adjuvants, such as sucrose, lactose or starch. These compositions can comprise substances other than diluents, for example a lubricant, such as magnesium stearate, or a coating intended for controlled release.

Use can be made, as liquid compositions for oral administration, of solutions which are pharmaceutically acceptable, suspensions, emulsions, syrups and elixirs containing inert diluents, such as water or liquid paraffin. These compositions can also comprise substances other than diluents, for example wetting, sweetening or flavoring products.

In certain embodiments, the oral dosage forms are solid and prepared under anhydrous conditions with anhydrous ingredients, as described in detail in the sections above. However, the scope of the dosage forms extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions is typically present in from about 50 to about 99 weight percent of the pharmaceutical composition or dosage form.

Disintegrants are used in the compositions provided herein to provide tablets that disintegrate when exposed to an aqueous environment. Tablets that contain too much disintegrant can disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant. Disintegrants that can be used in pharmaceutical compositions and dosage forms provided herein include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. If used at all, lubricants are typically used in an amount of less than about 1 weight percent of the pharmaceutical compositions or dosage forms into which they are incorporated.

### Parenteral Dosage Forms

The compositions for parenteral administration can be emulsions or sterile solutions. Use can be made, as solvent or vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing and stabilizing agents. Sterilization can be carried out in several ways, for example using a bacteriological filter, by radiation or by heating. They can also be prepared in the form of sterile solid compositions that can be dissolved at the time of use in sterile water or any other injectable sterile medium.

### Other Dosage Forms

The compositions for rectal administration are suppositories or rectal capsules that contain, in addition to the active principle, excipients such as cocoa butter, semi-synthetic glycerides or polyethylene glycols.

The compositions can also be aerosols. For use in the form of liquid aerosols, the compositions can be stable sterile solutions or solid compositions dissolved at the time of use in apyrogenic sterile water, in saline or any other pharmaceutically acceptable vehicle. For use in the form of dry aerosols intended to be directly inhaled, the active principle is finely divided and combined with a water-soluble solid diluent or vehicle, for example dextran, mannitol or lactose.

In one embodiment, a composition disclosed herein is a pharmaceutical composition or a single unit dosage form. Pharmaceutical compositions and single unit dosage forms provided herein comprise a therapeutically effective amount of one or more therapeutic agents (e.g., a compound of formula (I), or other therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, and the like. In certain embodiments, water is a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Remington's Pharmaceutical Sciences, 16^{th}, 18^{th} and 20^{th} eds., Mack Publishing, Easton Pa. (1980, 1990 & 2000).

Typical pharmaceutical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Whether a particular excipient is suitable for incorporation into a pharmaceutical composition or dosage form depends on a variety of factors well known in the art including, but not limited to, the way in that the dosage form will be administered to a subject and the specific active ingredients in the dosage form. The composition or single unit dosage form, if desired, can also contain minor amounts of wetting or emulsifying agents, or pH buffering agents.

Compositions provided herein can be lactose free and comprise excipients that are well known in the art and are listed, for example, in the U.S. Pharmocopia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

In an embodiment, provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are, in certain embodiments, anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Exemplary embodiments further encompass pharmaceutical compositions and dosage forms that comprise one or more compounds that reduce the rate by which an active ingredient will decompose. Such compounds, that are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations, and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in one embodiment, in purified form, together with a suitable amount of carrier so as to provide the form for proper administration to the subject. The formulation should suit the mode of administration. In one embodiment, the pharmaceutical compositions or single unit dosage forms are sterile and in suitable form for administration to a subject, such as an animal subject, in one embodiment, a mammalian subject, such as a human subject.

A pharmaceutical composition provided herein is formulated to be compatible with its intended route of administration.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Typical dosage forms comprise a compound disclosed herein, or a pharmaceutically acceptable salt thereof lie within the range of from about 50 mg to about 1500 mg per day, given as a single once-a-day dose in the morning or as divided doses throughout the day. The composition can in certain embodiments be taken with food. In certain embodiments, dosage forms have about 50, about 100, about 200, about 250, about 300, about 400, about 500, about 600, about 750, or about 1000 mg of the compound of formula (I). The dosage forms can contain other amounts of a compound of formula (I) depending upon the results of additional testing.

In certain embodiments, the drug can be administered using intravenous infusion, an implantable osmotic pump, a transdermal patch, liposomes, or other modes of administration. In one embodiment, a pump can be used (see, Sefton, 1987, CRC Crit. Ref Biomed. Eng. 14: 201; Buchwald et al., 1980, Surgery 88: 507; Saudek et al., 1989, N. Engl. J. Med. 321: 574). In another embodiment, polymeric materials can be used. In yet another embodiment, a controlled release system can be placed in a subject at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Langer, 1990, Science 249: 1527-1533). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the subject.

### Delayed Release Dosage Forms

Active ingredients such as the compounds of formula (I) can be administered by controlled release means or by delivery devices that are well known to those of ordinary skill in the art. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein. Thus, provided herein are single unit dosage forms suitable for oral administration such as, but not limited to, tablets, capsules, gelcaps, and caplets that are adapted for controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

### Parenteral Dosage Forms

Although solid, anhydrous oral dosage forms can be used, provided herein are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are, in one embodiment, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms provided herein.

### Transdermal, Topical & Mucosal Dosage Forms

In one embodiment, solid, anhydrous oral dosage forms can be used. In another aspect, provided herein are transdermal, topical, and mucosal dosage forms. Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16^{th}, 18^{th} and 20^{th} eds., Mack Publishing, Easton Pa. (1980, 1990 & 2000); and Introduction to Pharmaceutical Dosage Forms, 4^{th} ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, that can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, that are non toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th}, 18^{th} and 20^{th} eds., Mack Publishing, Easton Pa. (1980, 1990 & 2000).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

### Methods of Treating or Preventing Disease in a Subject

The compounds of formula (I) can act on enzymes called cyclophilins and inhibit their catalytic activity. Accordingly, in another aspect, provided herein are methods to inhibit cyclophilins comprising administering a compound or composition disclosed herein, for example, a compound of formula (I), or a composition comprising a compound of formula (I), to a subject in need thereof. Cyclophilins occur in a wide variety of different organisms, including human, yeast, bacteria, protozoa, metazoa, insects, plants, or viruses. In the case of infectious organisms, inhibition of the cyclophilin catalytic activity by compounds provided herein often results in an inhibitory effect on the organism. Furthermore, in humans the catalytic activity of cyclophilins plays a role in many different disease situations. Inhibition of this catalytic activity is often associated to a therapeutic effect. Therefore, certain compounds described herein can be used for the treatment of infections including those caused by HCV, HBV and HIV.

Methods of Treating or Preventing HCV, HBV and/or HIV Infection in a Subject

### Hepatitis C

Hepatitis is a disease defined by the inflammation of the liver. The symptoms of hepatitis include jaundice, anorexia (poor appetite) and malaise. Hepatitis is acute when it lasts less than six months and chronic when it persists longer. Chronic hepatitis can lead to cirrhosis of the liver and liver cancer, that results in liver failure and the need for liver transplantation.

A group of viruses known as the hepatitis viruses cause most cases of hepatitis worldwide. Hepatitis C is caused by the infection of the hepatitis C virus (HCV). HCV is divided into six different genotypes, genotypes 1-6, with multiple subtypes in each genotype class. Genotype I is the most common hepatitis C genotype in the United States, and is the most difficult to treat.

### Hepatitis B

There are more than 350 million people worldwide who are chronically infected with HBV. After a two to three month incubation period in which the host is unaware of the infection, HBV infection can lead to acute hepatitis and liver damage, including cirrhosis, liver failure and hepatocellular carcinoma. Most neonates and children under the age of 5 as well as 5% of the adult population exposed to HBV develop chronic infection. Persistent infection is the result of an inadequate immune response to HBV and continuous HBV replication is the key driver of immune-mediated liver injury and disease progression. HBV can cause fulminant hepatitis, a rapidly progressive, frequently fatal form of the disease in which sections of the liver are destroyed. Current treatment of HBV is with interferons or direct-acting agents such as synthetic nucleosides. There exists a need for new therapeutic agents that can improve the limitations of the existing therapies, which include the development of resistance to nucleosides, rebound of HBV replication after ceasing therapy, and the low rates of HBsAg clearance.

The persistence of DNA replication is facilitated by the HBV covalently closed circular DNA (cccDNA) in the nucleus. The cccDNA has an essential function in the HBV life cycle and is required for the production of viral proteins and to generate the RNA template for the HBV DNA polymerase. Finite pegylated interferon-□ (IFN-□) therapy or long term treatment with oral nucleos(t)ide analogs inhibiting HBV-DNA polymerase controls HBV DNA replication in most patients and improves hepatitis symptoms. There is, however, a need for novel strategies and therapeutic targets since the current therapies do not eliminate cccDNA and have demonstrated poor efficiency in inducing HBsAg loss and HBsAg seroconversion.

Cure of chronic hepatitis B requires the restoration of an anti-HBV adaptive immune response to eliminate functional cccDNA in hepatocytes and to generate neutralizing antibodies against HBsAg. The virus-host interactions determining the development of HBV-specific immune control or chronicity of infections have not been characterized. Persistent infection is highlighted by the lack of an effective CD4+ and CD8+ T cell response.

Several HBV proteins can interfere with the innate and adaptive immune response to HBV. They block the production of type 1 interferons in response to Toll-like receptor (TLR) ligands, inhibit interferon responsiveness and impair the innate and adaptive immune functions of dendritic cells (DCs). HBV and HBsAg interfere with the production of IFN-α and anti-viral cytokines by TLR9-activated plasmacytoid DCs (pDCs) and inhibit the antigen presentation by myeloid DCs.

Cyclophilins, in particular cyclophilin A (CypA), associate with HBsAg (Tian et al., 2010, J. Virol.; 84:3373). Exemplary compounds herein, which are CypA inhibitors, can ameliorate the tolerogenic effects of HBsAg and restore DC function to enhance innate and adaptive anti-HBV immune responses. Similar to its immunomodulatory role in HCV the CypA inhibitor can restore interferon responsiveness in infected hepatocytes. Thus, exemplary compounds herein, in combination with pegylated IFN-α or an antiviral nucleos(t)ide can restore immune control of HBV infection.

### Cyclophilin Inhibitors

Cyclophilins are a family of enzymes that assist in the folding and transportation of other proteins synthesized within a cell. Protein folding or misfolding plays a central role in the pathophysiology of a number of serious diseases, such as viral diseases, central nervous system disorders, cancer and cardiovascular diseases. Cyclophilin inhibitors, such as cyclosporine A, have been used for decades for the prophylaxis of organ rejection in transplant patients.

Cyclosporine A and certain derivatives have been reported as having anti-HCV activity, see Watashi et al., 2003, Hepatology 38:1282-1288, Nakagawa et al., 2004, Biochem. Biophys. Res. Commun. 313:42-7, and Shimotohno and K. Watashi, 2004, American Transplant Congress, Abstract No. 648 (American Journal of Transplantation 2004, Volume 4, Issue s8, Pages 1-653). Cyclosporine derivatives having HCV activity are described in International Patent Publication Nos. WO2005/021028, WO2006/039668, WO2006/038088, WO2007/041631, WO2008/069917, WO2010/002428, WO2010/076329, WO2010/088573. Cyclophilin inhibitors have been evaluated for use in the treatment of HCV include alisporivir ([8-(N-methyl-D-alanine),9-(N-ethyl-L-valine)]cyclosporine, also known as Debio 025), (melle-4)cyclosporine (also known as NIM-811) and 3-[(R)-2-(N,N-dimethylamino)ethylthio-sarcosine]-4-(gamma-hydroxymethylleucine)cyclosporine (also known as SCY-635).

Exemplary embodiments provide methods of using a compound or composition disclosed herein, for example, a compound of formula (I), or a composition comprising a compound of formula (I), for the treatment or prevention of a viral infection in a subject in need thereof. The methods generally comprise the step of administering to the subject an effective amount of the compound or composition to treat or prevent the viral infection. In certain embodiments, the viral infection is an HCV infection, an HBV or an HIV infection, or an HCV, HBV and HIV co-infection.

In certain embodiments, the subject can be any subject infected with, or at risk for infection with, HCV. Infection or risk for infection can be determined according to any technique deemed suitable by the practitioner of skill in the art. In certain embodiments, subjects are humans infected with HCV.

The HCV can be any HCV known to those of skill in the art. There are at least six genotypes and at least 50 subtypes of HCV currently known to those of skill in the art. The HCV can be of any genotype or subtype known to those of skill. In certain embodiments, the HCV is of a genotype or subtype not yet characterized. In certain embodiments, the subject is infected with HCV of a single genotype. In certain embodiments, the subject is infected with HCV of multiple subtypes or multiple genotypes.

In certain embodiments, the methods or compositions are administered to a subject following liver transplant. Hepatitis C is a leading cause of liver transplantation in the U.S., and many subjects that undergo liver transplantation remain HCV positive following transplantation. In one aspect, methods of treating such recurrent HCV subjects with a compound or composition disclosed herein are provided. In other embodiments, methods of treating a subject before, during or following liver transplant to prevent recurrent HCV infection are provided.

In certain embodiments, the subject can be any subject infected with, or at risk for infection with, HIV. Infection or risk for infection can be determined according to any technique deemed suitable by the practitioner of skill in the art. In certain embodiment, subjects are humans infected with HIV. The HIV can be any HIV known to those of skill in the art.

In certain embodiments, the subject has never received therapy or prophylaxis for HIV infection. In further embodiments, the subject has previously received therapy or prophylaxis for HIV infection. For instance, in certain embodiments, the subject has not responded to HIV therapy. In certain embodiments, the subject can be a subject that received therapy but continued to suffer from viral infection or one or more symptoms thereof. In certain embodiments, the subject can be a subject that received therapy but failed to achieve a sustained virologic response.

Certain embodiments provide methods of treating a subject that is refractory to treatment for HIV. For instance, in some embodiments, the subject can be a subject that has failed to respond to treatment with one or more therapeutic agents for HIV. In some embodiments, the subject can be a subject that has responded poorly to treatment with one or more therapeutic agents for HIV.

In certain embodiments, the subject has, or is at risk for, co-infection of HCV with HIV. For instance, in the United States, 30% of HIV subjects are co-infected with HCV and evidence indicates that people infected with HIV have a much more rapid course of their hepatitis C infection. Maier and Wu, 2002, World J Gastroenterol 8: 577-57. The methods provided herein can be used to treat or prevent HCV infection in such subjects. It is believed that elimination of HCV in these subjects will lower mortality due to end-stage liver disease. Indeed, the risk of progressive liver disease is higher in subjects with severe AIDS-defining immunodeficiency than in those without. See, e.g., Lesens et al., 1999, J. Infect. Dis. 179: 1254-1258. Advantageously, compounds of the provided herein have been shown to suppress HIV in HIV subjects. See, e.g., U.S. Pat. Nos. 5,977,067, 5,994,299, 5,948,884, and 6,583,265, and International Patent Publication Nos. WO99/32512 and WO99/67280, the contents of which are hereby incorporated by reference in their entireties. Thus, in certain embodiments, provided herein are methods of treating or preventing HIV infection and HCV infection in subjects in need thereof.

### Dosage and Unit Dosage Forms

In human therapeutics, the doctor will determine the posology which he considers most appropriate according to a preventive or curative treatment and according to the age, weight, stage of the infection and other factors specific to the subject to be treated. Generally, doses are from about 50 to about 1500 mg per day for an adult, from about 50 to about 500 mg per day, or from about 100 to about 750 mg per day for an adult.

In further aspects, provided herein are methods of treating or preventing HIV and/or HCV infection in a subject by administering, to a subject in need thereof, an effective amount of a compound disclosed herein, or a pharmaceutically acceptable salt thereof, with a high therapeutic index against HIV and/or HCV. The therapeutic index can be measured according to any method known to those of skill in the art, such as the method described in the examples below. In certain embodiments, the therapeutic index is the ratio of a concentration at which the compound is toxic, to the concentration that is effective against HIV and/or HCV. Toxicity can be measured by any technique known to those of skill including cytotoxicity (e.g., IC₅₀ or IC₉₀) and lethal dose (e.g., LD₅₀ or LD₉₀). Likewise, effective concentrations can be measured by any technique known to those of skill including effective concentration (e.g., EC₅₀ or EC₉₀) and effective dose (e.g., ED₅₀ or ED₉₀). In certain embodiments, similar measurements are compared in the ratio (e.g., IC₅₀/EC₅₀, IC₉₀/EC₉₀, LD₅₀/ED₅₀ or LD₅₀/ED₉₀). In certain embodiments, the therapeutic index can be as high as 2.0, 5.0, 10.0, 15.0, 20.0, 25.0, 50.0, 75.0, 100.0, 125.0, 150.0 or higher.

The amount of the compound or composition which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a subject is administered multiple dosages of a composition disclosed herein, not all of the dosages need be the same. For example, the dosage administered to the subject may be increased to improve the prophylactic or therapeutic effect of the composition or it may be decreased to reduce one or more side effects that a particular subject is experiencing.

In certain aspects, the provided unit dosages comprising a compound disclosed herein, or a pharmaceutically acceptable salt thereof, in a form suitable for administration. Such forms are described in detail above. In certain embodiments, the unit dosage comprises about 25 to about 1500 mg active ingredient. In particular embodiments, the unit dosages comprise about 50, about 100, about 125, about 250, about 500, about 750, or about 1500 mg active ingredient. Such unit dosages can be prepared according to techniques familiar to those of skill in the art.

### Combination Therapy

In certain embodiments, a compound disclosed herein is administered in combination with one second agent. In further embodiments, a second agent is administered in combination with two second agents. In still further embodiments, a second agent is administered in combination with two or more second agents.

Suitable second agents include small-molecule, orally bioavailable inhibitors of the HCV enzymes, nucleic-acid-based agents that attack viral RNA, agents that can modulate the host immune response. Exemplary second agents include: (i) current approved therapies (peg-interferon plus ribavirin), (ii) HCV-enzyme targeted compounds, (iii) viral-genome-targeted therapies (e.g., RNA interference or RNAi), and (iv) immunomodulatory agents such as ribavirin, interferon (IFN) and Toll-receptor agonists.

In certain embodiments, the second agent is a modulator of the NS3-4A protease. The NS3-4A protease is a heterodimeric protease, comprising the amino-terminal domain of the NS3 protein and the small NS4A cofactor. Its activity is essential for the generation of components of the viral RNA replication complex.

Examples of useful NS3-4A protease include telaprevir (Vertex/Janssen/Mitsubishi), boceprevir (Merck & Co.), simeprevir (Johnson & Johnson), ABT-450 (Abbott), ACH-1625 (Achillion), asunaprevir (BMS), BI-201335 (Boehringer-Ingelheim), GS-9451 (Gilead), danoprevir (Roche) and MK-5172 (Merck & Co).

In certain embodiments, the second agent is a modulator of the HCV NS5B The RNA-dependent RNA polymerase (nucleoside polymerase inhibitors). Examples of nucleoside polymerase inhibitors include GS-7977 (Gilead), INX-189 (BMS), mericitabine (Roche), IDX-184 (Idenix) and ALS-2200 (Vertex). In further embodiments, the second agent is a non-nucleoside modulator of NS5B. Examples of useful non-nucleoside modulators of NS5B include ABT-333 (Abbott), BMS-791325 (BMS), BI-217 (Boehringer-Ingelheim), tegobuvir (Gilead), setrobuvir (Roche) and VX-222 (Vertex).

In further embodiments, the second agent is a non-nucleoside modulator of NS5A. Examples of useful non-nucleoside modulators of NS5B include ABT-267 (Abbott), daclatasvir (BMS), GS-5885 (Gilead), ACH-3102 (Achillion) and IDX-719 (Idenix).

In a further embodiment, the second agent is an agent that modulates the subject's immune response. For instance, in certain embodiments, the second agent can be a presently approved therapy for HCV infection such as an interferon (IFN), a pegylated IFN, an IFN plus ribavirin or a pegylated IFN plus ribavirin. In certain embodiments, the interferons include IFNα, IFNα2a and IFNα2b, and particularly pegylated IFNα2a (PEGASYS®) or pegylated IFNα2b (PEG-INTRON®).

In a further embodiment, the second agent is a modulator of a Toll-like receptor (TLR). It is believed that TLRs are targets for stimulating innate anti-viral response. Suitable TLRs include, but are not limited to, TLR3, TLR7, TLR8 and TLR9. It is believed that toll-like receptors sense the presence of invading microorganisms such as bacteria, viruses and parasites. They are expressed by immune cells, including macrophages, monocytes, dendritic cells and B cells. Stimulation or activation of TLRs can initiate acute inflammatory responses by induction of antimicrobial genes and pro-inflammatory cytokines and chemokines.

In certain embodiments, methods of administering a compound of formula (I) in combination with a second agent effective for the treatment or prevention of HIV infection are provided. The second agent can be any agent known to those of skill in the art to be effective for the treatment of FIIV infection. The second agent can be presently known or later developed.

In certain embodiments, methods of administering a compound of formula (I) in combination with a second agent effective for the treatment or prevention of HBV infection are provided. The second agent can be any agent known to those of skill in the art to be effective for the treatment of HBV infection. The second agent can be presently known or later developed. Examples of second HBV agents include interferons, such as interferon alfa-2b and pegylated interferon alfa-2a; HBV therapeutic vaccine; antibody treatment; or a HBV direct antiviral agent, meaning an agent that interferes with specific steps in the hepatitis B virus (HBV) replication cycle. A direct antiviral agent that inhibits HBV replication may be for example any of the currently anti-HBV agents approved for the treatment of HBV, namely telbivudine, lamivudine, emtricitabine, entecavir, adefovir, clevudine and tenofovir.

In certain embodiments, the second agent can be formulated or packaged with the compounds of formula (I). Of course, the second agent will only be formulated with a compound of formula (I) when, according to the judgment of those of skill in the art, such co-formulation should not interfere with the activity of either agent or the method of administration. In certain embodiment, the compound of formula (I) and the second agent are formulated separately. They can be packaged together, or packaged separately, for the convenience of the practitioner of skill in the art.

The dosages of the second agents are to be used in the combination therapies. In certain embodiments, dosages lower than those which have been or are currently being used to prevent or treat infection are used in the combination therapies. The recommended dosages of second agents can be obtained from the knowledge of those of skill. For those second agents that are approved for clinical use, recommended dosages are described in, for example, Goodman & Gilman's The Pharmacological Basis Of Basis Of Therapeutics 9^{th} ed., Hardman et al., eds., Mc-Graw-Hill, New York (1996); Physician's Desk Reference (PDR) 57 ed., Medical Economics Co., Inc., Montvale, N.J. (2003), the contents of which are hereby incorporated by reference in their entireties.

In certain embodiments, the compound of formula (I) and the second agent are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, a compound of formula (I) and a second agent are administered to a patient, for example, a mammal such as a human, in a sequence and within a time interval such that the compound of formula (I) can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In one embodiment, the compound of formula (I) and the second active agent exert their effects at times which overlap. Each second active agent can be administered separately, in any appropriate form and by any suitable route. In other embodiments, the compound of formula (I) is administered before, concurrently or after administration of the second active agent.

In certain embodiments, the compound of formula (I) and the second agent are cyclically administered to a patient. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improve the efficacy of the treatment.

In other embodiments, courses of treatment are administered concurrently to a patient, i.e., individual doses of the second agent are administered separately yet within a time interval such that the compound of formula (I) can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the compound of formula (I). In one embodiment, a compound of formula (I) is administered concurrently with one or more second agents in the same pharmaceutical composition. In another embodiment, a compound of formula (I) is administered concurrently with one or more second agents in separate pharmaceutical compositions. In still another embodiment, a compound of formula (I) is administered prior to or subsequent to administration of a second agent. In one aspect, provided herein is administration of a compound of formula (I) and a second agent by the same or different routes of administration, e.g., oral and parenteral. In certain embodiments, when a compound of formula (I) is administered concurrently with a second agent that potentially produces adverse side effects including, but not limited to, toxicity, the second active agent can advantageously be administered at a dose that falls below the threshold that the adverse side effect is elicited.

### Kits

Kits for use in methods of treatment of HIV and/or HCV infection and/or HBV infection are provided. The kits can include a pharmaceutical compound or composition disclosed herein and instructions providing information to a health care provider regarding usage for treating or preventing a bacterial infection. Instructions may be provided in printed form or in the form of an electronic medium such as a floppy disc, CD, or DVD, or in the form of a website address where such instructions may be obtained. A unit dose of a compound or composition disclosed herein can include a dosage such that when administered to a subject, a therapeutically or prophylactically effective plasma level of the compound or composition can be maintained in the subject for at least 1 day. In some embodiments, a compound or composition disclosed herein can be included as a sterile aqueous pharmaceutical composition or dry powder (e.g., lyophilized) composition. In one embodiment, the compound is according to formula (I).

In some embodiments, suitable packaging is provided. As used herein, “packaging” refers to a solid matrix or material customarily used in a system and capable of holding within fixed limits a compound or composition disclosed herein suitable for administration to a subject. Such materials include glass and plastic (e.g., polyethylene, polypropylene, and polycarbonate) bottles, vials, paper, plastic, and plastic-foil laminated envelopes, and the like. If e-beam sterilization techniques are employed, the packaging should have sufficiently low density to permit sterilization of the contents.

The kits may also comprise, in addition to the compound or composition disclosed herein, second agents or compositions comprising second agents for use with the compound or composition as described in the methods above.

### Synthesis

The following Examples illustrate the synthesis of representative compounds of formula (I) using Intermediates 1-15 which illustrate the synthesis of intermediates. These examples are not intended, nor are they to be construed, as limiting the scope of the embodiments disclosed herein. It will be clear that various embodiments may be practiced otherwise than as particularly described herein. Numerous modifications and variations are possible in view of the teachings herein and, therefore, are within the scope.

### Synthesis of Intermediates Used to Produce Compounds of Formula (I)

### Intermediate 1

### Preparation of methyl 1-[(tert-butoxycarbonyl)amino]cyclopropanecarboxylate

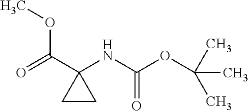

Di-t-butyl dicarbonate (671.4 g, 3.08 mole) was added to methyl 1-aminocyclopropanecarboxylate hydrochloride (453.1 g, 2.99 mole) in methylene chloride (2700 mL) cooled below 4° C. Sodium hydroxide (2M, 1700 mL) was added at such a rate to maintain a temperature below 7° C. The reaction was held for 18 hours and sodium chloride was added (200 g). The reaction was then held for an additional 18 hours. After the hold period, the phases were separated. The aqueous phase was extracted with methylene chloride (500 mL). The combined organic extracts were dried with anhydrous sodium sulfate filtered and concentrated. The concentrate was then slurried in heptane (600 mL). The solids were filtered and washed with heptane. The filtrate was concentrated to provide 284 g, which was then filtered and washed with heptane. The combined solids were dried in vacuum oven to provide Intermediate 1.

### Intermediate 2

### Preparation of methyl 1-[(tert-butoxycarbonyl)methylamino]cyclopropanecarboxylate

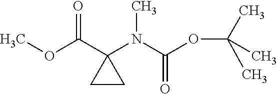 [SMILES: COC(=O)C1(N(C)C(=O)OC(C)(C)C)CC1]

Sodium bis trimethylsilyl amide (1M, 3250 mL) solution was added to a solution of methyl 1-[(tert-butoxycarbonyl)amino]cyclopropanecarboxylate (Intermediate 1) (536.4 g, 2.49 mole) in anhydrous tetrahydrofuran (1500 mL) keeping the temperature below 5° C. The reaction was held 1 hour and then the reaction mixture was cooled to below −10° C. Methyl iodide (530 g, 3.73 mole) was added at such a rate to maintain a temperature below −10° C. The reaction was allowed to warm to room temperature and held overnight. Ammonium chloride solution (15%, 1400 mL) was added and the mixture was agitated for 2 hours. Agitation was discontinued and the phases were allowed to separate. The organic phase was then concentrated. Methylene chloride (2500 mL) was added to the residue and the organic layer was washed with 20% ammonium chloride solution (1000 mL) for 30 minutes. The phases were separated, and the organic phase was extracted with 20% ammonium chloride (1500 mL) for 1 hour. The phases were separated, and the dichloromethane was extracted with 20% ammonium chloride (1500 mL) for 18 hours. The phases were then separated, and the organic phase was concentrated. Toluene (150 mL) was added to the residue and the solution was concentrated to provide Intermediate 2.

### Intermediate 3

### Preparation of tert-butyl[1-(hydroxymethyl)cyclopropyl]carbamate

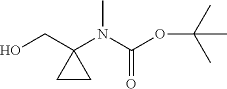

Sodium bis(2-methoxyethoxy)aluminum hydride (65%, 931 g, 2.99 mole) was added to a stirring solution of methyl 1-[(tert-butoxycarbonyl)methylamino]cyclopropanecarboxylate (Intermediate 2) (602 g, 2.99 mole) in toluene (1500 mL) at such a rate to maintain a temperature below 40° C. and held overnight. The reaction was cooled to 4° C. and 2N NaOH (1250 mL) was added at less than 15° C. Agitation was discontinued and the phases were separated. The organic phase was dried over anhydrous sodium sulfate, filtered and concentrated to provide Intermediate 3.

### Intermediate 4

### Preparation of 1-{[(tert-butoxycarbonyl)amino]cyclopropyl}methyl methanesulfonate

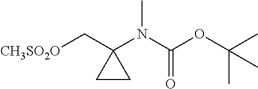

Methane sulfonyl chloride (125.9 g, 1.10 mole) was added to a stirring solution of tert-butyl[1-(hydroxymethyl)cyclopropyl]carbamate (Intermediate 3) (200.9 g, 0.999 mole) in methylene chloride (1000 mL) and triethylamine (111.1 g, 1.10 mole) at such a rate to maintain a temperature below 10° C. The reaction was held for 30 minutes after completion of the addition and water (700 mL) was added and stirred for 30 minutes. The phases were separated and the organic phase was dried with anhydrous sodium sulfate filtered and concentrated to provide Intermediate 4.

### Intermediate 5

### Preparation of tert-butyl[1-(bromomethyl)cyclopropyl]carbamate

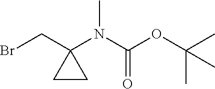

Lithium bromide (694 g, 7.99 mole) was added incrementally to a stirring solution of 1-[(tert-butoxycarbonyl)amino]cyclopropyl}methyl methanesulfonate (Intermediate 4) (300.5 g, 0.999 mole) in acetone (3000 mL) keeping the temperature less than 30° C. The reaction was held at room temperature for 18 hours. The reaction mixture was concentrated. Methylene chloride (2500 mL) was added followed by water to make the aqueous phase the top layer. The phases were separated and the organic layer was washed with water (700 mL). The organic layer was washed with anhydrous sodium sulfate filtered and concentrated to provide Intermediate 5.

### Intermediate 6

### Preparation of S-({(1-[(tert-butoxycarbonyl)amino]cyclopropyl}methyl) 4-methylbenzenesulfonothioate

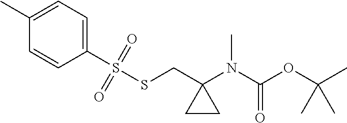 [SMILES: Cc1ccc(S(=O)(=O)SCC2(N(C)C(=O)OC(C)(C)C)CC2)cc1]

p-Toluenethiosulfonic acid potassium salt (333.5 g, 1.47 mole), tert-butyl[1-(bromomethyl)cyclopropyl]carbamate (Intermediate 5) (259.3 g, 0.982 mole), and 18 crown 6 (25.9 g, 0.0981 mole) in acetonitrile (2500 mL) was heated to 77° C. under nitrogen for 15 hours. Upon cooling, the reaction mixture was filtered and concentrated. The concentrate was dissolved in methylene chloride (1500 mL) and the organic layer was extracted with saturated sodium bicarbonate solution (500 mL). The organic layer was dried over anhydrous sodium sulfate, filtered and concentrated to provide Intermediate 6.

### Intermediate 7

### Preparation of S-{[1-(methylamino)cyclopropyl]methyl}-4-methylbenzenesulfonothioate hydrochloride

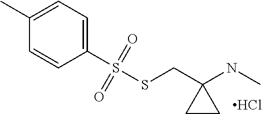

Hydrochloric acid in dioxane (4N, 730 mL) was added to S-({1-[(tert-butoxycarbonyl)amino]cyclopropyl}methyl) 4-methylbenzenesulfonothioate (Intermediate 6) (392.2 g, 0.982 mole) in dioxane (400 mL) keeping the temperature below 31° C. After 3.5 hours, methyl tert-butyl ether (MTBE) (1000 mL) was added slowly over 1 hour and the solid was filtered and washed with MTBE. The solid was dried in a vacuum oven at 50° C. to provide Intermediate 7.

### Intermediate 8

### Preparation of S-{[1-(dimethylamino)cyclopropyl]methyl}-4-methylbenzenesulfonothioate

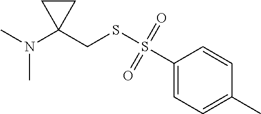

Triethylamine (131 g, 1.30 mole) was added to S-{[1-(methylamino)cyclopropyl]methyl}-4-methylbenzenesulfonothioate hydrochloride (Intermediate 7) (252.3 g, 0.866 mole) in methylene chloride (2500 mL) keeping the temperature below 5° C. Aqueous formaldehyde (37%, 105.5 g, 1.30 mole) was then added over 5 minutes. After holding for 30 minutes, sodium triacetoxyborohydride (315.6 g, 1.50 mole) was added incrementally keeping the temperature <6° C. After 1.5 hours, saturated sodium bicarbonate (1250 mL) was added and agitated for 10 minutes. The phases were separated. The organic layer was dried over anhydrous sodium sulfate, filtered and concentrated to provide Intermediate 8, ¹H NMR (400 MHz, CDCl₃) δ 0.55 (m, 2H) 0.72 (m, 2H) 2.26 (s, 6H) 2.46 (s, 3H) 3.18 (s, 2H) 7.34 (d, 2H J=8.2 Hz) 7.79 (d, 2H J=8.2 Hz).

### Intermediate 9

### Preparation of methyl 1-aminocyclopropanecarboxylate hydrochloride

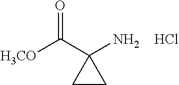

Thionyl chloride (887 g, 7.46 moles) was added to a stirred slurry of 1-aminocyclopropane carboxylic acid in methanol (5000 mL) at such a rate to maintain a temperature below 32° C. An ice bath was used to cool the reaction. On completion of the addition, the reaction mixture was heated to reflux for 5 hours. On cooling, the solvent was removed and the resulting solid was slurried in MTBE (1000 mL). The slurry was filtered, washed with MTBE, and dried to give to provide Intermediate 9 as a white solid.

### Intermediate 10

### Preparation of methyl 1-isopropylaminocyclopropane carboxylate

Triethylamine (241 g, 2.38 mole) was added to a stirred slurry of methyl 1-aminocyclopropanecarboxylate hydrochloride (Intermediate 10) (240.7 g, 1.59 mole) and acetone (120 g, 2.07 mole) in methylene chloride (3500 mL) under nitrogen keeping the temperature below 7° C. The reaction was held for 30 minutes and sodium triacetoxyborohyldride (438 g, 2.07 mole) was added, keeping the temperature in the 10-15° C. range. The reaction was held at 15° C. for 3 hours after completion of the addition. Saturated aqueous sodium bicarbonate (750 mL) was added over 30 minutes and agitated for 1 hour. The phases were then separated and the organic layer was extracted with saturated sodium bicarbonate solution (500 mL). After phase separation, the organic layer was dried with anhydrous sodium sulfate, filtered and concentrated to provide Intermediate 10.

### Intermediate 11

### Preparation of methyl 1-(N-isopropyl-N-methylamino)cyclopropane carboxylate

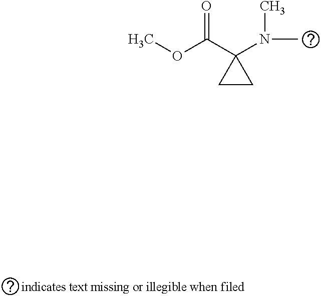

A solution of methyl 1-isopropylaminocyclopropane carboxylate (Intermediate 10) (249 g, 1.58 mole) in methylene chloride (3000 mL) with aqueous formaldehyde (37%, 167 g, 2.06 mole) and acetic acid (6 mL) was stirred at room temperature under nitrogen for 1.5 hours. On cooling below 9° C., sodium triacetoxyborohydride (437 g, 2.06 mole) was added incrementally over 1.5 hours keeping the temperature below 12° C. The reaction was held for 2 hours after completion of the addition. After the hold period, the reaction was quenched with saturated sodium bicarbonate (1000 mL) and the phases separated. The organic layer was then extracted with saturated sodium bicarbonate (750 mL). The phases were separated and the organic phase was dried with anhydrous sodium sulfate, filtered and concentrated to provide Intermediate 11 as a white solid (85.9%).

### Intermediate 12

### Preparation of 1-(N-isopropyl-N-methylamino)cyclopropanemethanol hydrochloride.

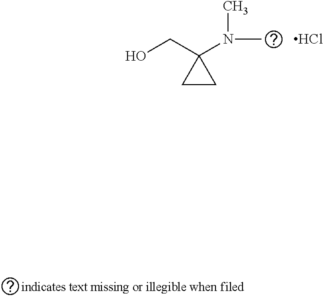

Sodium (bis 2-methoxyethoxy)aluminum hydride (506 g, 1.64 mole) was added dropwise to a solution of methyl 1-(isopropyl-methylamino)cyclopropane carboxylate (Intermediate 11) (232 g, 1.36 mole) in toluene (2500 mL) keeping the temperature below 27° C. The reaction was held for 18 hours at ambient temperature. Cooled to below 5° C. and 2 M NaOH (750 mL) was added, keeping the temperature below 10° C. The phases were separated and the organic phase was dried with anhydrous sodium sulfate and filtered. To the filtrate was added 5-6 N HCl in isopropanol (300 mL) and concentrate the solvent to half the volume. The solid was filtered and washed with MTBE (500 mL). The product was dried in a vacuum oven to provide Intermediate 12.

### Intermediate 13

### Preparation of 1-chloromethyl-1-(N-isopropyl-N-methylamino)cyclopropane hydrochloride

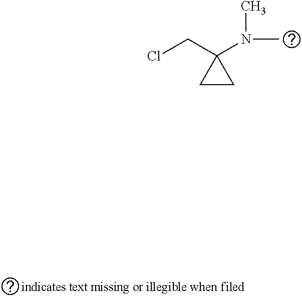

Thionyl chloride (183 g, 1.54 mole) was added dropwise over 1.5 hour to a stirring slurry of 1-(N-isopropyl-N-methylamino)cyclopropanemethanol hydrochloride (Intermediate 12) (229.5 g, 1.28 mole) in toluene (2400 mL) at 50° C. The reaction was held for 1 hour after completion of the addition. The reaction was then allowed to cool to room temperature. The batch was then concentrated to approximately 900 g. The solid was then filtered and washed with MTBE (500 mL). The product was dried in a vacuum oven at 50° C. to provide Intermediate 13.

### Intermediate 14

### Preparation of S-({1-[methyl(propan-2-yl)amino]cyclopropyl}methyl) 4-methylbenzenesulfonothioate

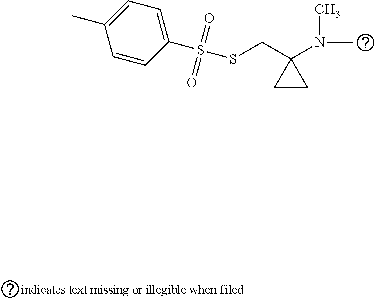

Potassium carbonate (79.89 g, 0.578 mole) followed by p-toluenethiosulfonic acid potassium salt (139.22 g, 0.623 mole) were added to a stirring slurry of 1-chloromethyl-1-(isopropylmethylamino)cyclopropane hydrochloride (Intermediate 13) (110.94 g, 0.560 mole) in acetonitrile (1100 mL). The reaction mixture was stirred at ambient temperature overnight. The resulting slurry was filtered through Celite and concentrated. The concentrate was dissolved in methylene chloride (1000 mL) and extracted twice with saturated sodium bicarbonate solution (200 mL). The organic layer was dried over anhydrous sodium sulfate and concentrated to provide Intermediate 14, ¹H NMR (400 MHz, CDCl₃) δ 0.56 (m, 2H), 0.75 (m, 2H) 0.97 (d, 6H J=6.4 Hz) 2.25 (s, 3H) 2.46 (s, 3H) 2.82 (m, 1H) 3.19 (s, 2H) 7.34 (d, 2H J=8.2 Hz) 7.79 (d, 2H J=8.2 Hz).

[4′-Hydroxy-N-methylleucine]⁴cyclosporine A was prepared according to the method described in European Patent No. 484,281; and [4′-hydroxy-N-methylleucine]⁴-(N-benzyl)-Val⁵-cyclosporine A was prepared according the methods disclosed in WO2009/148615, the disclosures of which are specifically incorporated by reference in their entireties. The later compound is described in Papageorgiou et al, Bioorganic & Medicinal Chemistry (1997), Volume 5(1), pages 187-192.

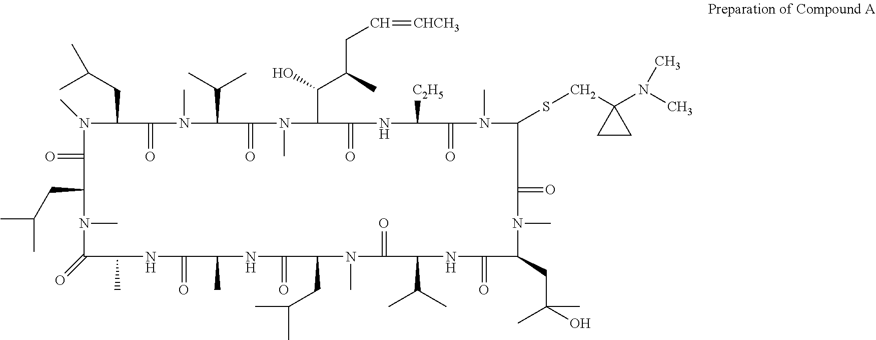

A 12 L jacketed cylindrical reactor was charged with anhydrous tetrahydrofuran (THF) (2.2 L) and diisopropylamine (DIPA; 142 mL, 1013 mmol, 13 equiv.) and stirred for 30 minutes. The water content was measured via Karl-Fischer coulombic titration (174 ppm) and cooled to −40° C. To this solution was added n-BuLi (405 mL, 1013 mmol, 13 equiv.) over 10 minutes (max temperature during addition was −30° C.). This solution was stirred for 30 minutes at −40° C., at which time a solution of [4′-hydroxy-N-methylleucine]-4-cyclosporine A (93.6 g, 76.8 mmol) was added over 15 minutes (max temperature during addition was −30° C.). This mixture was held at −40° C. for 2 hours, at which time S-{[1-(dimethylamino)cyclopropyl]methyl}-4-methylbenzenesulfonothioate (145 g, 506 mmol, 6.6 equiv) in THF was added over 10 minutes (max temperature during addition was −32° C.) and the temperature was raised to −25° C. over 1 hour. The mixture was held at −25° C. for 1 h and the temperature was raised to 0° C. over 1 hour and quenched with glacial acetic acid (125 mL, 28 equiv.) and stirred at room temperature overnight. To the mixture was then added water (1.0 L), and the mixture was stirred for 30 minutes and the phases split. The organic (top) layer was concentrated to minimal volume to give a viscous oil which was reconstituted in MTBE (1.0 L) and water (1.0 L). The mixture was stirred for 30 minutes and the phases split. To the organic layer (top) was added water (0.5 L) followed by ammonium hydroxide (0.5 L of a 30% aq. solution) so that the final pH1 was between 11-12. This mixture was stirred for 14 h at which time no electrophile was detected (HPLC). The phases were again spilt and the organic layer stripped to minimum volume and this residue was chromatographed and eluted with mobile phase A=heptanes, B=5% MeOH/EtOAc: (0-100% B, ˜50 L total MPA+MPB used) to give after removal of solvent, 106 g of material. This material was dissolved in 1 L MTBE and 1 L water and stirred 30 min, the aqueous layer was discarded and to the organic 1.0 L water added and the pH adjusted to 2.5+0.2 with 1 N HCl. The mixture was stirred 15 min, the phases allowed to spilt for 15 minutes, and the organic layer was discarded and the aqueous layer washed four more times with MTBE. During this time, it may be necessary to break emulsions with up to 2% v/v saturated brine solution. To the aqueous layer was then added MTBE (1.0 L) and the pH was adjusted to 9-10 with ammonium hydroxide. After stirring for 15 minutes, the organic layer was concentrated to provide the crude material (81 g). Final purification was achieved through silica gel chromatography (methanol/dichloromethane; 1.5 kg silica cartridge) to provide Compound A as a solid, ¹H NMR (400 MHz, CDCl₃) 0.5-0.6 (m, 2H), 0.6-1.1 (m, 39H), 1.1-1.8 (m, 23H), 1.35 (d, J=7.3 Hz, 3H), 1.9-2.2 (m, 4H), 2.3-2.5 (m, 3H), 2.35 (s, 6H), 2.70 (s, 6-1), 2.74 (d, J=12.9 Hz, 1H), 2.83 (d, J=12.9 Hz, 1H), 3.12 (s, 3H), 3.17 (s, 3H), 3.25 (s, 3H), 3.44 (s, 3H), 3.50 (s, 3H), 3.63 (d, J=6 Hz, 1H), 3.75 (q, J=6.4 Hz, 1H), 4.54 (quintet, J=7.4 Hz, 1H), 4.6-4.7 (m, 1H), 4.84 (quintet, J=7.0 Hz, 1H), 4.9-5.0 (m, 1H), 5.0-5.1 (m, 1H), 5.13 (d, J=10.9 Hz, 1H), 5.2-5.6 (m, 5H), 5.70 (dd, J=10.7, 4.2 Hz, 1H), 5.81 (s, 1H), 7.15 (d, J=8.0 Hz, 1H), 7.48 (d, J=8.3 Hz, 1H), 7.64 (d, J=7.7 Hz, 1H), 7.93 (d, J=9.7 Hz, 1H).

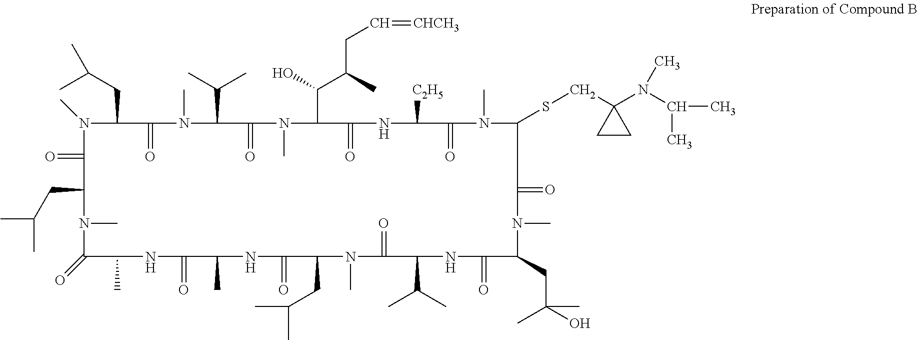

A 12 L jacketed cylindrical reactor was charged with anhydrous THF (2.2 L) and diisopropylamine (DIPA; 140 mL, 1000 mmol, 13 equiv) and stirred for 30 minutes. The water content was measured via Karl-Fischer coulombic titration (174 ppm) and cooled to −40° C. To this solution was added n-BuLi (401 mL, 1000 mmol, 13 equiv) over 10 minutes (max temperature during addition was −30° C.). This solution was stirred for 30 minutes at −40° C., at which time a solution of [4′-hydroxy-N-methylleucine]-4-cyclosporine A (93.6 g, 76.8 mmol) was added over 15 minutes (max temperature during addition was −30° C.). This mixture was held at −40° C. for 2 hours, at which time S-({1-[methyl(propan-2-yl)amino]cyclopropyl}methyl) 4-methylbenzenesulfonothioate (158 g, 504 mmol, 6.6 equiv) in 158 mL THF was added over 10 minutes and the temperature was raised to −25° C. over 1 hour. The mixture was held at −25° C. for 1 hour and the temperature was raised to 0° C. over 1 hour and quenched with glacial acetic acid (125 mL, 28 equiv) and stirred at room temperature overnight. To the mixture was then added water (1.0 L), and the mixture stirred for 30 minutes and the phases split. The organic (top) layer was stripped to minimal volume to give a viscous oil which was reconstituted in MTBE (1.0 L) and water (1.0 L) and the mixture stirred for 30 minutes and the phases split and to the organic layer (top) was added water (0.5 L) followed by ammonium hydroxide (0.5 L of a 30% aq. solution) so that the final pH was between 11-12. This was stirred for 14 h, and the aqueous layer discarded. Water (0.5 L) and ammonium hydroxide (0.5 L) were then added so that the pH is >12. This mixture was stirred further for 6 h, at which time no electrophile was detected (HPLC). The phases were again split and the organic layer concentrated to a minimum volume. The crude material was further purified by successive silica gel chromatography (elution with ethyl acetate/heptanes for the first column and then methanol/dichloromethane for the 2^{nd} column) to provide Compound B as a solid; ¹H NMR (500 MHz, CDCl₃) 0.5-1.1 (m, 47H), 1.2-1.8 (m, 23H), 1.34 (d, J=7.3 Hz, 3H), 2.0-2.2 (m, 5H), 2.3-2.5 (m, 3H), 2.34 (m, 3H), 2.69 (s, 6H), 2.76 (d, J=12.8 Hz, 1H), 2.82 (d, J=15.9 Hz, 1H), 3.11 (s, 3H), 3.16 (s, 3H), 3.24 (s, 3H), 3.42 (s, 3H), 3.49 (s, 3H), 3.6 (m, 1H), 3.74 (q, J=6.2 Hz, 1H), 4.53 (quintet, J=7.4 Hz, 1H), 4.6-4.7 (m, 1H), 4.83 (quintet, J=7.3 Hz, 1H), 4.9-5.0 (m, 1H), 5.0-5.1 (m, 2H), 5.12 (d, J=10.8 Hz, 1H), 5.25-5.45 (m, 3H), 5.47 (d, J=6.2 Hz, 1H), 5.6-5.7 (m, 1H), 5.75 (s, 1H), 7.14 (d, J=8.0 Hz, 1H), 7.47 (d, J=8.4 Hz, 1H), 7.63 (d, J=7.6 Hz, 1H), 7.91 (d, J=9.5 Hz, 1H).

By proceeding according to method described above for the synthesis of Compounds A or Compound B the following compounds were also prepared:

[(R)-[(1-(N,N-dimethylamino)cyclobutyl)methylthio-Sar]³[4′-hydroxy-N-methylleucine]⁴-cyclosporine A (Compound C), ¹H NMR (400 MHz, CHCl₃-d) δ ppm 0.70 (d, 2H) 0.93 (m, 30H) 1.09 (d, J=6.49 Hz, 2H) 1.30 (m, 13H) 1.48 (m, 2H) 1.76 (m, 12H) 2.09 (m, 4H) 2.39 (m, 15H) 2.70 (d, J=1.46 Hz, 4H) 2.94 (s, 2H) 3.03 (m, 2H) 3.13 (s, 2H) 3.18 (s, 2H) 3.25 (s, 2H) 3.49 (d, J=10.35 Hz, 4H) 3.76 (m, J=10.18, 2.31, 1.20, 1.20 Hz, 1H) 4.54 (m, 1H) 4.64 (dd, J=9.40, 8.61 Hz, 1H) 4.84 (m, 1H) 4.98 (m, 1H), 5.07 (m, 1H) 5.13 (d, J=10.93 Hz, 1H) 5.35 (m, 1H) 5.43 (m, 1H) 5.51 (d, J=5.91 Hz, 1H) 5.70 (m, 1H) 5.97 (s, 1H) 7.15 (d, J=8.05 Hz, 1H) 7.53 (d, J=8.35 Hz, 1H) 7.62 (d, J=7.76 Hz, 1H) 7.95 (d, J=9.52 Hz, 1H) 8.28 (m, 1H).

[(R)-[(1-(N,N-diethylamino)cyclopropyl)methylthio-Sar]³[4′-hydroxy-N-methylleucine]-4-cyclosporine A (Compound D), ¹H NMR (400 MHz, CHCl₃-d) δ ppm 0.60 (d, 2H) 0.70 (d, J=6.00 Hz, 3H) 0.90 (m, 40H) 1.27 (m, 15H) 1.59 (m, 14H) 2.08 (m, 4H) 2.45 (m, 3H) 2.70 (d, J=1.76 Hz, 6H) 2.79 (d, J=7.56 Hz, 1H) 3.02 (m, 1H) 3.12 (s, 3H) 3.17 (s, 3H) 3.25 (s, 3H) 3.44 (s, 3H) 3.49 (m, 3H) 3.60 (m, 1H) 3.75 (m, J=17.25, 0.88, 0.63, 0.63 Hz, 1H) 4.55 (quin, J=7.38 Hz, 1H) 4.64 (dd, J=9.22, 8.88 Hz, 1H) 4.84 (qd, J=7.27, 7.03 Hz, 0H) 4.98 (dd, J=9.00, 6.95 Hz, 1H) 5.07 (t, J=7.42 Hz, 1H) 5.13 (d, J=10.93 Hz, 1H) 5.35 (m, 3H) 5.50 (d, J=6.05 Hz, 1H) 5.70 (dd, J=10.81, 4.17 Hz, 1H) 5.74 (s, 1H) 7.15 (d, J=7.96 Hz, 1H) 7.49 (d, J=8.15 Hz, 1H) 7.62 (d, J=7.47 Hz, 1H) 7.94 (d, J=9.71 Hz, 1H).

[(R)-[(1-(N-ethyl-N-methylamino)cyclopropyl)methylthio-Sar]³[4′hydroxy-N methylleucine]-4-cyclosporine A (Compound E), ¹H NMR (400 MHz, CHCl₃-d) δ ppm 0.60 (m, 1H) 0.70 (dddd, J=19.52, 1.02, 0.84, 0.72 Hz, 1H) 0.77 (m, 2H) 0.92 (m, 29H) 1.10 (m, 3H) 1.25 (d, J=17.86 Hz, 6H) 1.36 (m, 3H) 1.62 (m, 22H) 2.08 (m, 4H) 2.34 (d, J=11.76 Hz, 0H) 2.45 (m, 3H) 2.62 (m, 2H) 2.70 (s, 3H) 2.81 (m, 2H) 3.01 (m, 1H) 3.11 (m, 3H) 3.18 (m, 2H) 3.27 (m, 2H) 3.44 (m, 2H) 3.53 (d, J=1.22 Hz, 0H) 3.62 (m, 1H) 3.76 (m, 1H) 4.51 (m, 1H) 4.64 (m, 1H) 4.84 (m, 1H) 5.06 (m, 3H) 5.35 (m, 3H) 5.50 (m, 1H) 5.70 (m, 1H) 5.78 (m, =13.58, 1.79, 1.79, 1.05 Hz, 0H) 7.15 (m, 1H) 7.49 (m, 1H) 7.65 (m, 1H) 7.94 (m, 1H).

[(R)-[(1-(N,N-dimethylamino)cyclobutyl)methylthio-Sar]³-(N-benzyl)-Val⁵ cyclosporine A (Compound F), ¹H NMR (400 MHz, DMSO-d₆) δ ppm 0.40 (s, 2H) 0.83 (m, 35H) 1.07 (m, 2H) 1.15 (m, 3H) 1.31 (m, 4H) 1.50 (m, 1H) 1.62 (m, 8H) 2.09 (s, 5H) 2.80 (m, 5H) 2.88 (s, 2H) 2.95 (s, 2H) 3.07 (s, 2H) 3.19 (s, 2H) 3.33 (m, 16H) 4.12 (m, 1H) 4.29 (m, 1H) 4.59 (m, 1H) 4.66 (d, J=4.34 Hz, 1H) 4.72 (m, 1H) 4.91 (d, J=10.54 Hz, 1H) 5.02 (m, 1H) 5.14 (dddd, J=9.08, 1.68, 0.76, 0.54 Hz, 2H) 5.26 (dd, J=3.95, 0.78 Hz, 1H) 5.39 (m, 3H) 6.34 (d, J=0.20 Hz, 1H) 6.55 (dt, J=7.77, 0.56 Hz, 1H) 7.02 (m, 1H) 7.27 (m, 3H) 8.17 (dd, J=7.69, 1.24 Hz, 1H) 8.39 (ddd, J=1.04, 0.68, 0.57 Hz, 1H) 8.47 (m, 1H).

### HCV Activity

The compounds of formula (I) were tested for activity against HCV using methods adapted from those described by Kriger et al., 2001, Journal of Virology 75: 4614-4624, Pietschmann et al., 2002, Journal of Virology 76: 4008-4021, and using HCV RNA constructs as described in U.S. Pat. No. 6,630,343. Compounds were examined in the human hepatoma cell a HCV RNA replicon containing a stable luciferase (LUC) reporter. The HCV RNA replicon ET contains the 5′ end of HCV (with the HCV Internal Ribosome Entry Site (IRES) and the first few amino acids of the HCV core protein) which drives the production of neomycin phosphotransferase (NeoR) fusion protein. A luciferase reported is incorporated into 1b replicon. The EMCV IRES element controls the translation of the HCV structural proteins NS3-NS5. The NS3 protein cleaves the HCV polyprotein to release the mature NS3, NS4A, NS4B, NS5A and NS5B proteins that are required for HCV replication. At the 3′ end of the replicon is the authentic 3′ NTR of HCV. The activity of the LUC reporter is directly proportional to HCV replication levels and positive-control antiviral compounds produce a reproducible antiviral response using the LUC endpoint.

The compounds were dissolved in DMSO at six half-log concentrations. HCV replicon cells were plated out into 96 well plates dedicated for the analysis of cell numbers (cytotoxicity) or antiviral activity and the next day the compounds were added to the appropriate wells. The cells were processed 72 hours later when the cells were still subconfluent. Antiviral activity was expressed as EC₅₀ and EC₉₀, the effective concentration of compound that reduced viral replication by 50% and 90%, respectively. Compound EC₅₀ and EC₉₀ values were derived from HCV RNA levels assessed as HCV RNA replicon derived LUC activity. Cytotoxicity was expressed as IC₅₀ and IC₉₀, the concentration of compound that inhibited cell viability by 50% and 90%, respectively. Compound IC₅₀ and IC₉₀ values were calculated using a colorimetric assay as an indication of cell numbers and cytotoxicity. The activity of the LUC reporter is directly proportional to HCV RNA levels in the human cell line. The HCV-replicon assay was validated in parallel experiments using interferon-alpha-2b as a control. The compounds were tested in 1a replicon, (qRT_PCR/TaqMan), 1b replicon (LUC) and 2a replicon (qRT-PCR/TaqMan). The following mean EC₅₀ values were obtained (in nM): Compound Genotype 1b

| Compound | Genotype 1b |
|---|---|
| A | 60 |
| B | 60 |
| C | 160 |
| D | 70 |
| E | 20 |
| F | 520 |

In addition Compound A demonstrated anti-HCV activity in other genotypes, with EC₅₀ values of 70 nM against Genotype 1a and 60 nM against genotype 2a.

### HBV Activity

The compounds of formula (I) are also tested for antiviral activity against the human Hepatitis B Virus (HBV) in a number of established hepatic cell lines either transiently transfected with a plasmid expressing HBV or stably transfected cell lines such as AD38 cells. In the transient assays, either HepG2 or HuH7 cells are transfected with a 1.1× unit length HBV genome (Durantel et al., 2004, Hepatology 40: 855-864), while for AD38 cells, HBV expression is induced via a tetracycline-inducible promoter. The cells are then incubated in the presence of various concentrations of test compounds for 4 or 7 days. At the end of the incubation, intracellular HBV DNA is isolated and quantitated either by real-time PCR or by Southern Blot analysis. Antiviral activity can also be evaluated by analysis of HBV particles secreted from the cells into the cell culture media either through analysis of the particle HBV DNA or by evaluation of HBeAg present in the cell supernatant. Antiviral activity is expressed as EC₅₀ and EC₉₀, the effective concentration of compound that reduced viral replication by 50% and 90%, respectively. Anti-HBV activity is also tested in the HepaRG cell line following infection with HBV. Following incubation of the infected cells in various concentration of test compound, including treatment of HepaRG cells prior to HBV infection, antiviral activity is determined by quantitation of either intracellular or extracellular HBV DNA by real-time PCR.

In this assay Compound A had an EC50 of >20 μM and Compound B had an EC50 of >20.1M.

### HIV Activity

The compounds of formula (I) are also tested for antiretroviral activity against human immunodeficiency virus-1 (HIV) using infection of the human T-lymphoblastoid cell line, CEM-SS, with the HIV strain HIV-1IIIB (Weislow et al., 1989, J. Natl. Cancer Inst. 81: 577-586). In this MTS cytoprotection assay, each experiment included cell control wells (cells only), virus control wells (cells plus virus), drug toxicity wells (cells plus drug only), drug colorimetric control wells (drug only) as well as experimental wells (drug plus cells plus virus). Compounds are first dissolved in DMSO and tested using six half-log dilutions, starting with a high concentration of either 20 or 2 μM. HIV-1RF is added to each well in a volume of 50 μL, the amount of virus determined to give approximately 90% cell killing at 6 days post-infection. At assay termination, assay plates are stained with the soluble tetrazolium-based dye MTS (CellTiter 96 Reagent, Promega) to determine cell viability and quantify compound toxicity. MTS is metabolized by the mitochondria enzymes of metabolically active cells to yield a soluble formazan product, providing a quantitative analysis of cell viability and compound cytotoxicity. The assay is validated in parallel experiments using Zidovudine (3′-azido-3′-deoxythymidine or AZT) as a positive control.

### Anti-Human Respiratory Syncytial Virus (RSV) Cytoprotection Assay:

Cell Preparation—

HEp2 cells (human epithelial cells, ATCC catalog #CCL-23) were passaged in DMEM supplemented with 10% FBS, 2 mM L-glutamine, 100 U/mL penicillin, 1001 g/mL streptomycin 1 mM sodium pyruvate, and 0.1 mM NEAA, T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 1×10⁴ cells per well in tissue culture medium and added to flat bottom microtiter plates in a volume of 100 μL. The plates were incubated at 37° C./5% CO₂ overnight to allow for cell adherence. Medium was then removed and drug added to the microtiter plates in a volume of 100 μL.

Virus Preparation—

The RSV strain Long and RSV strain 9320 were obtained from ATCC (catalog #VR-26 and catalog #VR-955, respectively) and were grown in HEp2 cells for the production of stock virus pools. A pretitered aliquot of virus was removed from the freezer (−80° C.) and allowed to thaw slowly to room temperature in a biological safety cabinet. Virus was resuspended and diluted into assay medium (DMEM supplemented with 2% heat-inactivated FBS, 2 mM L-glutamine, 100 U/mL penicillin, 100 μg/mL streptomycin, 1 mM sodium pyruvate, and 0.1 mM NEAA) such that the amount of virus added to each well in a volume of 100 μL was the amount determined to yield 85 to 95% cell killing at 6 days post-infection. In this assay Compound A demonstrated a 68% reduction in virus replication against RSV strain 9320 at 10 μg/ml.

### Anti-Influenza Virus Cytoprotection Assay

Cell Preparation—

MDCK cells (canine kidney cells, ATCC catalog #CCL-34) were passaged in DMEM supplemented with 10% FBS, 2 mM L-glutamine, 100 U/mL penicillin, 100 μg/mL streptomycin, 1 mM sodium pyruvate, and 0.1 mM NEAA, T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 1×10⁴ cells per well in tissue culture medium and added to flat bottom microtiter plates in a volume of 100 L. The plates were incubated at 37° C./5% CO₂ overnight to allow for cell adherence. Medium was then removed and the monolayers were washed with DPBS. The compound was then added to the microtiter plates in a volume of 100 μL.

Virus Preparation—

The influenza A/CA/05/09 (CDC), A/HK/8/68 (ATCC catalog #VR-544) and B/Allen/45 (ATCC catalog #VR-102) strains were obtained from ATCC or from the Center of Disease Control and were grown in MDCK cells for the production of stock virus pools. A pretitered aliquot of virus was removed from the freezer (−80° C.) and allowed to thaw slowly to room temperature in a biological safety cabinet. Virus was resuspended and diluted into assay medium (DMEM supplemented with 2 mM L-glutamine, 100 U/mL penicillin, 100 μg/mL streptomycin, 1 mM sodium pyruvate, 0.1 mM NEAA, and 1 μg/ml TPCK-treated trypsin) such that the amount of virus added to each well in a volume of 100 μL was the amount determined to yield 85 to 95% cell killing at 4 days post-infection.

Efficacy and Toxicity XTT—

Following incubation at 37° C. in a 5% CO₂ incubator, the test plates were stained with the tetrazolium dye XTT (2,3-bis(2-methoxy-4-nitro-5-sulfophenyl)-5-[(phenylamino)carbonyl]-2H-tetrazolium hydroxide). XTT-tetrazolium was metabolized by the mitochondrial enzymes of metabolically active cells to a soluble formazan product, allowing rapid quantitative analysis of the inhibition of virus-induced cell killing by antiviral test substances. XTT solution was prepared daily as a stock of 1 mg/mL in RPM11640. Phenazine methosulfate (PMS) solution was prepared at 0.15 mg/mL in PBS and stored in the dark at −20° C. XTT/PMS stock was prepared immediately before use by adding 40 L of PMS per ml of XTT solution. Fifty microliters of XTT/PMS was added to each well of the plate and the plate was reincubated for 4 hours at 37° C. Plates were sealed with adhesive plate sealers and shaken gently or inverted several times to mix the soluble formazan product and the plate was read spectrophotometrically at 450/650 nm with a Molecular Devices Vmax plate reader.

Data Analysis—

Raw data was collected from the Softmax Pro 4.6 software and imported into a Microsoft Excel XLfit 4 spreadsheet for four parameter curve fit analysis.

Compound F had the following levels of inhibition at 10 uM:

### Influenza A H1N1 (A/CA/05/09): 98%.

### Influenza A H3N2 (A/HK/8/68): 68%.

### Influenza B (B/Allen/45): 15%.

### Cyclophilin Binding Activity

The cyclophilin inhibition binding of the compounds of formula (I) was determined using a competitive ELISA adapted from the methods described by Quesniaux et al. (Quesniaux et al., 1987, Eur. J Immunol. 27: 1359-1365). Activated ester of succinyl spacers bound to D-Lys⁸-cylosporine A (D-Lys⁸-Cs) was coupled to bovine serum albumin (BSA) through D-lysyl residue in position 8. BSA was dissolved in 0.1 M borate buffer, pH 9.0 (4 mg in 1.4 ml). A hundredfold molar excess of D-Lys⁸-Cs dissolved in dimethyl formamide (0.6 ml) was added drop wise to the BSA under vigorous stirring. The coupling reaction was performed for 2 to 3 hours at room temperature under mild stirring and the conjugate was extensively dialyzed against phosphate-buffered saline (PBS, pH 7.4). After acetone precipitation of an aliquot of the conjugated protein, no covalently bound D-Lys⁸-Cs remained in the acetone solution and the extent of cyclosporine covalent binding was calculated.

Microtiter Plates were coated with D-Lys⁸-Cs-BSA conjugate (2 ggiml in PBS for 24 hours at 4° C.). Plates were washed with Tween®/PBS and three times with PBS alone. To block nonspecific binding, 2% BSA/PBS (pH 7.4) was added to the wells and allowed to incubate for 2 hours at 37° C. A five-fold dilution series of the compound to be tested was made in ethanol in a separate microtiter plate. The starting concentration was 0.1 mg/mL for assays with human recombinant cyclophilin. 198 μL of 0.1 μg/mL cyclophilin solution was added to the microtiter immediately followed by 2 L of diluted cyclosporine A (used as a reference compound) or a compound disclosed herein. The reaction between coated BSA-Cs conjugate, free cyclosporine A or a compound disclosed herein and cyclophilin was allowed to equilibrate overnight at 4° C. Cyclophilin was detected with anti-cyclophilin rabbit antiserum diluted in 1% BSA containing PBS and incubated overnight at 4° C. Plates were washed as described above. Bound rabbit antibodies were then detected by goat anti-rabbit IgG conjugated to alkaline phosphatase diluted in 1% BSA-PBS and allowed to incubate for 2 hours at 37° C. Plates were washed as described above. After incubation with 4-nitrophenyl phosphate (1 g/l in diethanolamine buffer, pH 9.8) for 1 to 2 hours at 37° C., the enzymatic reaction was measured spectrophotometrically at 405 nm using a spectrophotometer

The following IC50 values (nM) were obtained:

| Compound | Cyclophilin A | Cyclophilin B | Cyclophilin D |
|---|---|---|---|
| A | 20 | 13 | 598 |
| B | 18 | 23 | N/A |
| C | 12 | 11 | 610 |
| D | 71 | 64 | N/A |
| E | N/A | N/A | N/A |
| F | 57 | 43 | 395 |

### IL-2 Activity

Compounds of formula (I) were tested for their inhibition of IL-2 production by stimulated T Cells, using Jurkat cells with anti-CD3 and anti-CD28 co-stimulation. All compounds had a 0.5-Log 9-point titration starting at 10 μM (n=2) to 0.0015 μM. Cyclosporine A (control) was also run at a 0.5-Log 9-point titration. starting at 500 ng/mL. All compounds to be tested were dissolved in dimethyl sulfoxide. Cytotoxicity was evaluated with parallel Alamar Blue plates. Jurkat cells were seeded at 1×OS cells per well in 200 μL growth media in a 96-well plate. Cells were cultured in complete RPMI medium supplemented with 10% fetal bovine serum with one hour incubation at 37° C. with 5% carbon dioxide

PMA and PHA were diluted in complete media to a final concentration of 1 ng/mL and 5 μg/mL respectively. The diluted compounds were mixed 1:1 and 25 μL of the mixture was added per well. The plates were incubated overnight at 37° C. in 5% CO₂.

The following day one hundred microliters of cell culture supernatant were transferred from each well to a non-sterile V-bottom plate and stored plate at −80° C. prior to analysis. Determination of IL-2 concentrations in the supernatant was performed using the Pierce Human IL-2 Colorimetric ELISA kit as per the manufacturer's directions (Pierce, #EH2IL25). Cell culture supernatants were diluted with an equal volume of complete RPMI (25 μL sample+25 μL RPMI per well) prior to testing in order to remain in linear range of the assay.

Duplicate wells containing cells in the absence of PMA/PHA and test drug (background) and duplicate wells containing cells stimulated with PMA/PHA in the absence of test drug (100% production of IL-2) were included as controls. Activated cells treated with DMSO were used to normalize IL-2 values. IL-2 standard curves were generated using a 4-parameter curve fit in SoftMax Pro software from Molecular Devices.

Two independent assays were performed to evaluate the effect of cyclosporine A and representative compounds on IL-2 production in PMA/PHA stimulated Jurkat cells (mean values were calculated). IL-2 production was inhibited by 50% (IC₅₀) in the presence of 4.35 ng/mL cyclosporine A. The following IC₅₀ values were obtained (in ng/mL)

| | IL-2 inhibition |
|---|---|
| Compound | IC50 |
| A | 4842 |
| B | 7690 |
| C | 9310 |
| D | 13400 |
| E | >13620 |
| F | 452 |

### Determination of Cytokine Production in Human Peripheral Blood Mononuclear Cells (PBMCs)

Cryopreserved commercial or previously isolated PBMCs from HCV donors cryopreserved in RPMI1640)/Fetal Bovine/DMSO solution (50/40/10 v/v) were tested with compounds provided herein.

Whole blood tubes for IL28B genotyping were also drawn and the genotyping was performed using real time PCR with allele-specific Taqman probes to detect the single nucleotide polymorphism rs12979860 C/T on chromosome 18q13.

PBMC were cultured for 24 hours at 37° C. in RPMI cell culture medium in flat bottom 48 well-plates. Each well received 180 μL of cell suspension (2×10⁶ cells/mL). Treatments (20 μL) are added to each well (2 wells per condition), and include RPMI (control), RPMI with DMSO (0.005%), and the compound provided herein at a concentration of 20 μM in RPMI (for a final treatment of 2 μM cyclosporine A or compound provided herein). At the end of the incubation, the plates are centrifuged at 200 times g for 5 minutes. The cell supernatants are collected and assayed by ELISA for cytokines IFN-α's (the detection reagent recognized 14 of the 15 known human IFN-α subtypes) and IFN-λ1 (IL-29). The cell pellet is washed twice with cold PBS and ATP content are determined by adding 100 μL per well of the Cell Titer Glo® (Promega, Madison, Wis.). Plates are then placed at −80° C. until protein analysis. For the protein analysis, plates are thawed, scraped and the protein content of the cell suspension determined using BCA Protein Assay Kit (23227, ThermoScientific, Rockford Ill.).

Donor demographics are summarized in Table 1. The utilization of PBMC from these donors in different assay series is summarized in Table 2.

TABLE 1
Demographics and IL28B genotyping of the healthy and HCV positive
donors.

| | Infection | | | | IL28B genotype |
|---|---|---|---|---|---|
| Donor # | status | Age | Gender | Race | (rs12979860) |
| 1 | Health | 54 | Male | Hispanic/Black | CT |
| 2 | Healthy | 47 | Male | Black | TT |
| 3 | Healthy | NA | NA | NA | NA |
| CT1 | HCV | 57 | Female | Caucasian | CT |
| CT2 | HCV | 57 | Male | African | CT |
| | | | | American | |
| CT4* | HCV | 68 | Male | NA | CT |
| CC1 | HCV | 43 | Female | NA | CC |

NA, not available
*donor on Humalin treatment

TABLE 2
Assay Series Performed. Each series represents assays performed on the same day.

| Assay | | | Background in DMSO- |
|---|---|---|---|
| Series | Donor(s) | Comments | treated controls* |
| 1 | Healthy 1 | 400 μL supernatant, 400,000 cells/well | None detected |
| 1 | Healthy 2 | 400 μL supernatant, 400,000 cells/well | ~22 pg/mL IL29 |
| 2 | CT2 | First test of donor CT2, 200 μL supernatant, | None detected |
| | | 400,000 cells/well | |
| 3 | CT4 | Donor on Humalin treatment, 200 μL | ~4 pg/mL IFNα, ~6 pg/mL |
| | | supernatant, 400,000 cells/well | IL29 |
| 4 | CT1, CT2 | Second test of donor CT2, 200 μL | None detected |
| | | supernatant, 400,000 cells/well | |
| 4 | Healthy 3 | 200 μL supernatant, 400,000 cells/well, | None detected |
| | | cryopreserved | |
| 5 | CT2** | 3rd test of donor CT2; PBMCs | None detected |
| | | cryopreserved, 200 μL supernatant, 400,000 | |
| | | cells/well | |
| 6 | CC1 | 200 μL supernatant, 400,000 cells/well | None detected |

*Assayed markers detected in cell supernatants. These background values were subtracted from the levels detected following compound treatments of these cell batches.
**Cells from same collection as CT2 second test, tested following cryopreservation and thawing.

PBMC from three healthy donors were tested to determine whether they released interferons following treatment with compounds provided herein. DMSO-treated PBMC from one of these donors (#2) produced IL-29, and these levels increased slightly following treatment, (Table 3). PBMC from the other two donors did not produce IFN-α or IL-29 following any of the treatments (Tables 3 and 4).

PBMC from multiple HCV positive donors were tested in a series of independent assays for interferon responses following treatment with compounds provided herein (Table 2). Cells from all the HCV positive donors produced IFNα and IL-29 following treatment with compounds provided herein (Tables 3 and 4). In the table below LOQ means “limit of quantification”.

TABLE 3
IL-29 concentrations in PBMC supernatants following compound
treatment (2 μM for 24 hours).

| | Compound A | | | Compound B | | |
|---|---|---|---|---|---|---|
| Donor | pg/mL IL29 | | Av. | pg/mL IL29 | | Av. |
| CT1 | 13 | 17 | 15 | 17 | 17 | 17 |
| CT2 | 2307 | 2153 | 2230 | 1057 | 937 | 997 |
| CT2 (test 2) | 112 | 88 | 100 | 99 | 93 | 96 |
| CT2 (test 3) | 65 | 71 | 68 | 88 | 95 | 91.5 |
| CT4 | 9 | 8 | 8.5 | 11 | 21 | 16 |
| CC1 | 95 | 94 | 94.5 | 80 | 78 | 79 |
| Healthy 1 | <LOQ | <LOQ | <LOQ | 20 | <LOQ | 10 |
| Healthy 2 | 15 | 16 | 15.5 | <LOQ | <LOQ | <LOQ |
| Healthy 3 | <LOQ | <LOQ | <LOQ | <LOQ | <LOQ | <LOQ |

TABLE 4
IFN-α concentrations in PBMC supernatants following compound
treatment (2 μM for 24 hours).

| | Compound A | | | Compound B | | |
|---|---|---|---|---|---|---|
| Donor | pg/mL IFNα | | Av. | pg/mL IFNα | | Av. |
| CT1 | 17 | 13 | 15 | 18 | 23 | 20.5 |
| CT2 | 4 | 4 | 4 | 8 | 6 | 7 |
| CT2 (test 2) | 7 | 6 | 6.5 | 8 | 7 | 7.5 |
| CT2 (test 3) | 7 | 7 | 7 | 8 | 8 | 8 |
| CT4 | 5 | 3 | 4 | 6 | 4 | 5 |
| CC1 | 17 | 19 | 18 | 15 | 17 | 16 |
| Healthy 1 | <LOQ | <LOQ | <LOQ | <LOQ | <LOQ | <LOQ |
| Healthy 2 | <LOQ | <LOQ | <LOQ | <LOQ | <LOQ | <LOQ |
| Healthy 3 | <LOQ | <LOQ | <LOQ | <LOQ | <LOQ | <LOQ |

### Example B1

### Effect of Compounds on HBV-Mediated Inhibition of Interferon and Cytokine Induction by Toll-Like Receptor Agonists

PBMC from healthy donors are tested with compounds provided herein. In some experiments plasmacytoid or myeloid DC-depleted PBMC fractions are used. PBMC or PBMC fractions are stimulated with Toll-like receptor ligands in the presence or absence of HepAD38-derived HBV and compound in XVIVO 15 media in either 96- or 48-well tissue culture plates. Supernatants are collected 18 h after stimulation. Production of cytokines and interferons is determined by ELISA. The list of cytokines and interferons tested includes but is not limited to IFN-α, IFN-β, IFN-γ, IFN-λ, IL-6, IL-10, IL-12, IL-23 and TNF-α.

Plasmacytoid (pDCs) and myeloid dendritic cells (mDCs) are isolated from human PBMC of healthy donors using magnetic bead separation kits and are tested with compounds provided herein. PDCs or mDCs are stimulated with Toll-like receptor ligands in the presence or absence of HepAD38-derived HBV and compound in XVIVO 15 media in either 96- or 48-tissue culture plates. In some pDC experiments the XVIVO media contains IL-3. Supernatants are collected 18 h after stimulation. Production of cytokines and interferons is determined by ELISA. The list of cytokines and interferons tested includes but is not limited to IFN-α, IFN-β, IFN-γ, IFN-α, IL-6, IL-10, IL-12, IL-23 and TNF-α.

Monocyte-derived DCs (MoDCs) are generated from monocytes using IL-4 and GMCSF. Monocytes are purified from PBMC of healthy donors. MoDCs are tested with the compounds herein. MoDCs are stimulated with Toll like receptor ligands in the presence or absence of HepAD38-derived HBV and compound in XVIVO 15 media in 96-well tissue culture plates. Supernatants are collected 18 h after stimulation. Production of cytokines and interferons is determined by ELISA. The list of cytokines and interferons tested includes but is not limited to IFN-α, IFN-β, IFN-γ, IFN-λ, IL-6, IL-10, IL-12, IL-23 and TNF-α.

### Example B2

### Effect of Compounds on HBV-Mediated Modulation of Costimulatory Molecules Expression by PBMC and Dendritic Cells

PBMC from healthy donors are tested with compounds provided herein. Expression of costimulatory molecules such as CD40, CD80 and CD86 are determined by flow cytometry using an 8 colour FACSverse instrument. PBMC are stimulated with Toll-like receptor ligands in the presence or absence of HepAD38-derived HBV and compound in XVIVO 15 media in 6 well tissue culture plates. After 24 h cell will be block and stained with a set of fluorescence labelled antibodies including but not limited to antibodies against human CD1c, CD3, CD11c, CD14, CD19, CD20, CD40, CD80, CD86, CD123, CD141, CD303, CD304 and HLA-DR. The mean fluorescence intensity of CD40, CD)₈₋₀ and CD86 will be assessed by flow cytometry for HLADR+ cells and mDC1 (CD1c+), pDC(CD123+, CD303+ or CD123+, CD304+) and mDC2 (CD141⁺) subpopulations.

### Example B3

### Effect of Compounds on Cytokine and Interferon Production by PBMC from HBV-Infected Chimpanzee

PBMC from healthy and HBV-infected chimpanzee are tested with compounds provided herein. In some experiments plasmacytoid or myeloid DC-depleted PBMC fractions are used. PBMC or PBMC fractions are stimulated with Toll like receptor ligands in the presence or absence of compound in XVIVO 15 media in either 96-tissue culture plates. Supernatants are collected 18 h after stimulation. Production of cytokines and interferons is determined by ELISA. The list of cytokines and interferons tested includes but is not limited to IFN-α, IFN-β, IFN-γ, IFN-λ, IL-6, IL-10, IL-12, IL-23 and TNF-α.

### Example B4

### Effect of Compounds on the Expression of Costimulatory Molecules by PBMC and Dendritic Cells from HBV-Infected Chimpanzee

PBMC from healthy and HBV-infected chimpanzee are tested with compounds provided herein. Expression of costimulatory molecules such as CD40, CD80 and CD86 are determined by flow cytometry using an 8 colour FACSverse instrument. PBMC are stimulated with Toll-like receptor ligands in the presence or absence of compound in XVIVO 15 media in 6 well tissue culture plates. After 24 h cell will be block and stained with a set of fluorescence labelled antibodies including but not limited to antibodies against human CD1c, CD3, CD11c, CD14, CD19, CD20, CD40, CD80, CD86, CD123, CD141, CD303, CD304 and HLA-DR. The mean fluorescence intensity of CD40, CD80 and CD86 will be assessed by flow cytometry for HLADR+ cells and mDC1 (CD1c+), pDC(CD123+, CD303+ or CD123+, CD304+) and mDC2 (CD141+) subpopulations.

### Example B5

### Effect of Compounds on the Interferon Responsiveness of HBV-Expressing Cells

The human hepatoblastoma cell line HepAD38 expressing HBV is tested with the compounds provided herein. HepAD38 cells express HBV proteins and release HBV virions. The expression of HBV is induced by removing tetracycline from culture media. HepAD38 cells cultured with or without tetracycline, as well as the parent HepG2 cells is stimulated with IFN-α or IFN-β in the presence or absence of compounds. Induction of interferon inducible genes is determined by PCR, flow cytometry or ELISA, respectively.

### Mitochondrial Permeability Transition

Mitochondrial permeability transition (MPT) was determined by measuring swelling of the mitochondria induced by Ca²⁺. The procedure was adapted from the method described by Blattner et al., 2001, Analytical Biochem., 295: 220. Mitochondria were prepared from rat livers, which had been perfused with phosphate-buffered saline (PBS) to remove blood, using standard methods that utilized gentle homogenization in sucrose based buffer and then differential centrifugation to first remove cellular debris and then to pellet the mitochondria. Swelling was induced by 150 micro molar Ca²⁺ (added from a concentrated solution of Calcium chloride) and was monitored by measuring the scattering at 535-540 nm. Representative compounds were added 5 minutes before swelling was induced. EC₅₀ were determined by comparing swelling with and without the compounds of formula (I).

In the above test, Compound A gave an EC₅₀ value of 10 M or lower, indicating the ability of compounds of formula (I) to penetrate mitochondria and inhibit the MPT.

### Short Duration Treatment of Chronic Hepatitis C

Various methods related to the treatment and management of chronic hepatitis are described in detail below. The term “methods” include all the methods described herein, in particular the methods involving administering a compound of formula (I) in combination with interferon and optionally ribavirin.

In one aspect, provided herein is a method of modulating and/or sensitizing the immune system of a subject having chronic hepatitis C, such that the subject is responsive to interferon therapy or interferon/ribavirin therapy. Modulation of the immune system of a treated subject can be indicated by the induction of markers of the innate immune system, wherein an increase or decrease in the level of the markers of the innate immune system, as compared to the immune system of a subject undergoing interferon therapy or interferon/ribavirin therapy that has not been treated with a compound of formula (I), indicates that the immune system is being modulated. The method can include detecting and/or measuring the level of markers of the innate immune system to determine whether the immune system of a subject treated with a compound of formula (I) and undergoing interferon therapy or interferon/ribavirin therapy has been modulated.

In another aspect, disclosed herein is a method of inducing the sensitivity to interferon therapy or interferon/ribavirin therapy in a subject having chronic hepatitis C. The subject treated with a compound of formula (I) can have enhanced or improved sensitivity to interferon therapy or interferon/ribavirin therapy as compared to a subject that has not been treated with a compound of formula (I). The treated subject can experience an alleviation or amelioration of the symptoms caused by chronic HCV. The treated subject can have undetectable HCV RNA level or sustained undetectable HCV RNA as described below.

Also disclosed herein, is a method of inducing responsiveness to interferon therapy or interferon/ribavirin therapy in a subject having chronic hepatitis C. The subject treated with a compound of formula (I) can have enhanced or improved responsiveness to interferon therapy or interferon/ribavirin therapy as compared to a subject that has not been treated with a compound of formula (I). The treated subject can experience an alleviation or amelioration of the symptoms caused by chronic HCV. The treated subject can have undetectable HCV RNA level or sustained undetectable HCV RNA as described below.

In yet another aspect, disclosed herein is a method of inducing a sustained antiviral activity in a subject having chronic hepatitis after cessation of interferon therapy or interferon/ribavirin therapy. Antiviral activity can be sustained for greater than about five weeks, about 10 weeks, about 15 weeks, about 20 weeks, or about 24 weeks after cessation of treatment. Antiviral activity can be sustained for greater than about five weeks to about 24 weeks, for about 10 to 24 weeks, or for about 15 to 24 weeks after cessation of interferon therapy or interferon/ribavirin therapy. Sustained antiviral activity can be determined based on the level of HCV RNA present in the subject, such that a substantially undetectable level of HCV RNA in a subject indicates sustained antiviral activity. By “substantially undetectable level of HCV RNA,” it is understood to mean at a level of less than about 15 IU/mL.

In one embodiment, the methods provided herein can make the subject more susceptible to interferon therapy or interferon/ribavirin therapy, and the subject can experience an alleviation or amelioration in the symptoms associated with chronic hepatitis C.

In one embodiment, the methods provided herein are applied to subjects who never been treated with an interferon-based therapy.

In one embodiment, the methods provided herein are applied to subjects who have previously been treated with an interferon therapy but where therapy was unsuccessful. In one aspect of this embodiment the subject is a null responder, i.e. a person who achieved a less than 2 log 10 reduction in HCV RNA at week 12 of a prior course of therapy. In another aspect of this embodiment the subject is a prior relapser, defined as a person whose HCV RNA was undetectable at the completion of a prior course of therapy but whose hepatitis C virus became detectable during the follow-up period. In a further aspect of this embodiment the subject is a partial responder, defined as a person who achieved at least a 2 log 10 reduction in HCV RNA at week 12, but whose hepatitis C virus never became undetectable by week 24 of a prior course of therapy.

In one embodiment, the methods provided herein alleviate or ameliorate the symptoms associated with chronic hepatitis C. The term “alleviate” or “ameliorate” may refer to any indicia of success in the treatment of chronic hepatitis C, including any objective or subjective parameter such as abatement, remission or diminishing of symptoms or an improvement in a subject's physical well-being. Amelioration or alleviation of symptoms can be based on objective or subjective parameters; including the results of a physical examination. Some of the symptoms include but are not limited to jaundice, anorexia (poor appetite) and malaise.

In another embodiment, the methods described herein can include detecting and/or measuring the HCV RNA level to determine whether a subject is responsive or sensitive to interferon therapy or interferon/ribavirin therapy, at least one of before, during, and subsequent to cessation of the interferon therapy or interferon/ribavirin therapy, and whether a subject has sustained antiviral activity after cessation of the interferon therapy or interferon/ribavirin therapy. The methods can also include determining whether the subject is experiencing fewer symptoms associated with chronic hepatitis C relative to prior to starting treatment or symptoms of reduced severity.

In one embodiment, the methods provided herein include administering to a subject having chronic hepatitis C, effective amounts of a compound of formula (I), interferon, and optionally ribavirin for a short duration of time, such as about two weeks to six weeks. In another embodiment, the methods further comprise continued administration of interferon and optionally ribavirin for an additional about 20 weeks to about 52 weeks. The methods include the administration of the agents over two phases, an initial phase and a secondary phase. For instance, the initial phase can be a period of less than about six weeks and the secondary phase can be greater than or equal to about 20 weeks. The initial phase can be about two weeks to six weeks, and the secondary phase can be between about 20 to about 52 additional weeks. The initial phase can be about two, about three, about four, about five, or about six weeks, and the secondary phase can be about 20, about 24, about 28, about 32, about 36, about 40, about 44, about 48 or about 52 additional weeks. The initial phase can be about four weeks, and the secondary phase can be about 44 additional weeks. The secondary phase can follow immediately after the initial phase. The secondary phase can follow the initial phase after a brief interval of no treatment of about one day, about two days, about three days, about four days, about five days, about six days, about one week, or about two weeks. In the initial phase, a compound of formula (I) can be administered with interferon, and optionally with ribavirin. In the secondary phase, interferon can be administered by itself or optionally with ribavirin.

In one embodiment, in the initial phase of the treatment, the a compound of formula (I), interferon, and optionally ribavirin are administered for about two weeks to about six weeks, for example, for about four weeks, immediately followed by administration of interferon and optionally ribavirin for about 20 weeks to about 44 additional weeks in the secondary phase, for example, for about 44 additional weeks.

The methods provided herein can include a step of selecting for a subject with chronic hepatitis C. A “subject” can be any mammalian subject, such as a human subject. A subject to be treated by any of the methods described herein is an individual in need of treatment, such as a human subject. In some embodiments, the subject has been diagnosed with, or exhibits one or more symptoms of chronic hepatitis C. In other embodiments, the subject has been infected with HCV genotype 1.

In certain embodiments, the HCV is genotype 1HCV and can be of any subtype. For instance, in certain embodiments, the HCV is subtype 1a or 1b. It is believed that HCV infection of genotype 1 responds poorly to current interferon therapy. The methods provided herein can be advantageous for therapy of HCV infection with genotype 1. The methods provided herein can include a step of selecting for a subject infected with genotype 1HCV, in particular genotype 1a HCV.

In certain embodiments, the methods provided herein include a step involving selecting for subjects having chronic hepatitis C, selecting for subjects infected with HCV genotype 1, specifically genotype 1a, or selecting for subjects infected with HCV genotype 1, specifically genotype 1a and carrying a non CC genotype for the chromosome 19 single nucleotide polymorphism rs12979860. In certain embodiments, the methods provided herein include a step involving selecting for subjects infected with HCV genotype 1, specifically genotype 1a, and carrying an IL28 TT genotype or an IL28 CT genotype, for the chromosome 19 single nucleotide polymorphism rs12979860.

In one embodiment, the methods provided herein include administering to the subject having chronic hepatitis C, an effective amount of a compound of formula (I), as a divided dose in the course of an about 24 hour period, and in combination with interferon and optionally ribavirin.

It will be understood that, as used herein, references to amounts of a compound of formula (I) that have basic substituents refer to the amount of free base of the inhibitor.

In another embodiment, the methods include administering to a subject a pharmaceutical composition comprising an effective amount of a compound of formula (I) in combination with effective amounts of interferon and optionally ribavirin. In a further embodiment, the administration of a compound of formula (I) and optionally ribavirin can be made two or three times per day continually, for a number of days or weeks, and the administration of interferon can be made weekly or biweekly.

In a further embodiment, the methods include administering an effective amount of a compound of formula (I), in combination with other active agents, such as interferon and optionally ribavirin, wherein the three agents are administered to an infected subject in need thereof at least two times in an about 24 hour period, wherein each administration is preferably separated by about 8 to about 16 hours.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A cyclosporine A derivative in which the 3-Sarcosine position is substituted by a group —S—CH₂C[CH₂(CH₂)ₙ]NR²R³, wherein R² is hydrogen or an alkyl chain having from one to four carbon atoms and, when the alkyl chain has 3 or 4 carbon atoms, the chain is straight or branched; R³ is an alkyl chain having from one to four carbon atoms and, when the alkyl chain has 3 or 4 carbon atoms the chain is straight or branched; and n is 1 or 2.

2. A compound according to claim 1 of formula (I) 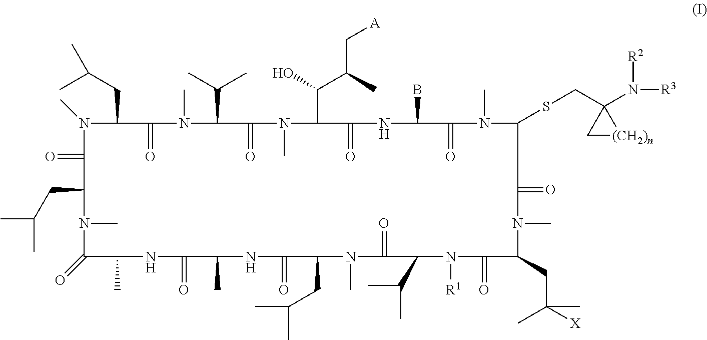
wherein:
A is (E) —CH═CHCH₃ or —CH₂CH₂CH₃;
B is ethyl, 1-hydroxyethyl, isopropyl or n-propyl;
n is 1 or 2;
X is hydroxyl or hydrogen;
R¹ is hydrogen or straight- or branched-chain alkyl containing from one to four carbon atoms optionally substituted by one or more groups R⁴ which may be the same or different;
R² is hydrogen or an alkyl chain having from one to four carbon atoms and, when the alkyl chain has 3 or 4 carbon atoms, the chain is a straight or branched; and
R³ is an alkyl chain having from one to four carbon atoms and, when the alkyl chain has 3 or 4 carbon atoms the chain is straight or branched;
R⁴ is phenyl optionally substituted by from one to five groups which may be the same or different selected from the group consisting of alkyl, haloalkyl, halogen, hydroxyl, alkoxy, amino, N alkylamino, N,N dialkylamino, carboxyl and alkoxycarbonyl;
or a pharmaceutically acceptable salt thereof.

3. The compound of claim 2, where X is hydroxyl.

4. The compound of claim 2, where A is (E) —CH═CHCH₃, B is ethyl, and n is 1.

5. The compound of claim 2, where A is (E) —CH═CHCH₃, B is ethyl, n is 1 and R² and R³ are each methyl.

6. The compound of claim 2, where
A is (E) —CH═CHCH₃;
B is ethyl;
n is 1 or 2;
R¹ is hydrogen or benzyl; and
R² and R³, which may be the same or different, each are a C₁-C₄ alkyl group.

7. The compound of claim 1, which is selected from the group consisting of:
[(R)-[(1-(N,N-dimethylamino)cyclopropyl)methylthio-Sar]³[4′hydroxy-N methylleucine]⁴ cyclosporine A;
[(R)-[(1-(N-methyl-N-isopropylamino)cyclopropyl)methylthio-Sar]³[4′hydroxy-N methylleucine]⁴ cyclosporine A;
[(R)-[(1-(N,N-dimethylamino)cyclobutyl)methylthio-Sar]³[4′hydroxy-N methylleucine]⁴ cyclosporine A;
[(R)-[(1-(N,N-diethylamino)cyclopropyl)methylthio-Sar]³[4′hydroxy-N methylleucine]⁴ cyclosporine A;
[(R)-[(1-(N-ethyl-N-methylamino)cyclopropyl)methylthio-Sar]³[4′hydroxy-N methylleucine]⁴-cyclosporine A; and
[(R)-[(1-(N,N-dimethylamino)cyclobutyl)methylthio-Sar]³-(N-benzyl)-Val⁵ cyclosporine A.

8. A composition comprising a compound of claim 1 and a pharmaceutically acceptable carrier.

9. A method of inhibiting cyclophilin, said method comprising administering an effective amount of a compound of claim 1.

10. A method of treating a subject infected with HBV, HCV or HIV, said method comprising administering an effective amount of a compound of claim 1 to said subject.

11. A method of inducing sensitivity to interferon therapy in a subject having chronic hepatitis C, the method comprising administering to the subject an effective amount of a combination of a compound of claim 1 with interferon for a period of about two weeks to about six weeks.

12. A compound of formula (III): 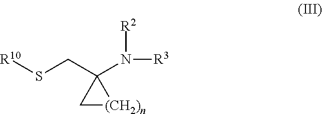
wherein R², R³ and n are as defined in claim 1 and R¹⁰ is a leaving group.
